SEQUENCE LISTING (32 sequences)

SEQ ID NO: 1 - protein, 735 aa, homo sapiens
HLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRR

SEQ ID NO: 2 - protein, 14 aa, homo sapiens
LGLYNLMNITRGSV

SEQ ID NO: 3 - protein, 13 aa, homo sapiens
KGKTNCPATVING

SEQ ID NO: 4 - protein, 13 aa, homo sapiens
WSKHNLTITQGKL

SEQ ID NO: 5 - protein, 20 aa, homo sapiens
NVTEFDGQDACGSNSWTVVD

SEQ ID NO: 6 - protein, 10 aa, homo sapiens
DIIYVQTDAT

SEQ ID NO: 7 - protein, 18 aa, homo sapiens
YEDSAGECCSCPKTDSQI

SEQ ID NO: 8 - protein, 620 aa, homo sapiens
SLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKIIIGPLIFVFLFSVVIGSIYLFLRKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPS

SEQ ID NO: 9 - protein, 10 aa, homo sapiens
KKNGRILTLP

SEQ ID NO: 10 - protein, 11 aa, homo sapiens
RILTLPRSNPS

SEQ ID NO: 11 - protein, 7 aa, homo sapiens
KNGRILT

SEQ ID NO: 12 - protein, 17 aa, homo sapiens
GGKKNGRILTLPRSNPS

SEQ ID NO: 13 - protein, 18 aa, homo sapiens
NGGKKNGRILTLPRSNPS

SEQ ID NO: 14 - protein, 1382 aa, homo sapiens
MATGGRRGAAAAPLLVAVAALLLGAAGHLYPGEVCPGMDIRNNLTRLHELENCSVIEGHLQILLMFKTRPEDFRDLSFPKLIMITDYLLLFRVYGLESLKDLFPNLTVIRGSRLFFNYALVIFEMVHLKELGLYNLMNITRGSVRIEKNNELCYLATIDWSRILDSVEDNYIVLNKDDNEECGDICPGTAKGKTNCPATVINGQFVERCWTHSHCQKVCPTICKSHGCTAEGLCCHSECLGNCSQPDDPTKCVACRNFYLDGRCVETCPPPYYHFQDWRCVNFSFCQDLHHKCKNSRRQGCHQYVIHNNKCIPECPSGYTMNSSNLLCTPCLGPCPKVCHLLEGEKTIDSVTSAQELRGCTVINGSLIINIRGGNNLAAELEANLGLIEEISGYLKIRRSYALVSLSFFRKLRLIRGETLEIGNYSFYALDNQNLRQLWDWSKHNLTITQGKLFFHYNPKLCLSEIHKMEEVSGTKGRQERNDIALKTNGDQASCENELLKFSYIRTSFDKILLRWEPYWPPDFRDLLGFMLFYKEAPYQNVTEFDGQDACGSNSWTVVDIDPPLRSNDPKSQNHPGWLMRGLKPWTQYAIFVKTLVTFSDERRTYGAKSDIIYVQTDATNPSVPLDPISVSNSSSQIILKWKPPSDPNGNITHYLVFWERQAEDSELFELDYCLKGLKLPSRTWSPPFESEDSQKHNQSEYEDSAGECCSCPKTDSQILKELEESSFRKTFEDYLHNVVFVPRKTSSGTGAEDPRPSRKRRSLGDVGNVTVAVPTVAAFPNTSSTSVPTSPEEHRPFEKVVNKESLVISGLRHFTGYRIELQACNQDTPEERCSVAAYVSARTMPEAKADDIVGPVTHEIFENNVVHLMWQEPKEPNGLIVLYEVSYRRYGDEELHLCVSRKHFALERGCRLRGLSPGNYSVRIRATSLAGNGSWTEPTYFYVTDYLDVPSNIAKIIIGPLIFVFLFSVVIGSIYLFLRKRQPDGPLGPLYASSNPEYLSASDVFPCSVYVPDEWEVSREKITLLRELGQGSFGMVYEGNARDIIKGEAETRVAVKTVNESASLRERIEFLNEASVMKGFTCHHVVRLLGVVSKGQPTLVVMELMAHGDLKSYLRSLRPEAENNPGRPPPTLQEMIQMAAEIADGMAYLNAKKFVHRDLAARNCMVAHDFTVKIGDFGMTRDIYETDYYRKGGKGLLPVRWMAPESLKDGVFTTSSDMWSFGVVLWEITSLAEQPYQGLSNEQVLKFVMDGGYLDQPDNCPERVTDLMRMCWQFNPKMRPTFLEIVNLLKDDLHPSFPEVSFFHSEENKAPESEELEMEFEDMENVPLDRSSHCQREEAGGRDGGSSLGFKRSYEEHIPYTHMNGGKKNGRILTLPRSNPS

SEQ ID NO: 15 - protein, 166 aa, homo sapiens
MKYTSYILAFQLCIVLGSLGCYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 16 - protein, 49 aa, homo sapiens
ILAFQLCIVLGSLGCYCQDPYVKEAENLKKYFNAGHSDVADNGTLFLGI

SEQ ID NO: 17 - protein, 143 aa, homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFRGRRASQ

SEQ ID NO: 18 - protein, 143 aa, homo sapiens
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFQGRRASQ

SEQ ID NO: 19 - protein, 55 aa, homo sapiens
QSQIVSFYFKLFKNFKDDQSIQKSVETIKEDMNVKFFNSNKKKRDDFEKLTNYSV

SEQ ID NO: 20 - protein, 46 aa, homo sapiens
MNVKFFNSNKKKRDDFEKLTNYSVTDLNVQRKAIHELIQVMAELSP

SEQ ID NO: 21 - protein, 39 aa, homo sapiens
SVETIKEDMNVKFFNSNKKKRDDFEKLTNYSVTDLNVQR

SEQ ID NO: 22 - protein, 25 aa, homo sapiens
VTDLNVQRKAIHELIQVMAELSPAA

SEQ ID NO: 23 - protein, 41 aa, homo sapiens
SYILAFQLCIVLGSLGCYCQDPYVKEAENLKKYFNAGHSDV

SEQ ID NO: 24 - protein, 21 aa, homo sapiens
ETIKEDMNVKFFNSNKKKRDD

SEQ ID NO: 25 - protein, 144 aa, homo sapiens
MQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELSPAAKTGKRKRSQMLFQGRRASQ

SEQ ID NO: 26 - protein, 144 aa, homo sapiens [sequence omitted]

SEQ ID NO: 27 - protein, 458 aa, homo sapiens
MNRGVPFRHLLLVLQLALLPAATQGKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

SEQ ID NO: 28 - protein, 47 aa, homo sapiens
IGLGIFFCVRCRHRRRQAERMSQIKRLLSEKKTCQCPHRFQKTCSPI

SEQ ID NO: 29 - protein, 36 aa, homo sapiens
GKKVVLGKKGDTVELTCTASQKKSIQFHWKNSNQIK

SEQ ID NO: 30 - protein, 15 aa, homo sapiens
DTYICEVEDQKEEVQ

SEQ ID NO: 31 - protein, 25 aa, homo sapiens
KEEVQLLVFGLTANSDTHLLQGQSL

SEQ ID NO: 32 - protein, 434 aa, homo sapiens
GKKVVLGKKGDTVELTCTASQKKSIQFHWKNSNQIKILGNQGSFLTKGPSKLNDRADSRRSLWDQGNFPLIIKNLKIEDSDTYICEVEDQKEEVQLLVFGLTANSDTHLLQGQSLTLTLESPPGSSPSVQCRSPRGKNIQGGKTLSVSQLELQDSGTWTCTVLQNQKKVEFKIDIVVLAFQKASSIVYKKEGEQVEFSFPLAFTVEKLTGSGELWWQAERASSSKSWITFDLKNKEVSVKRVTQDPKLQMGKKLPLHLTLPQALPQYAGSGNLTLALEAKTGKLHQEVNLVVMRATQLQKNLTCEVWGPTSPKLMLSLKLENKEAKVSKREKAVWVLNPEAGMWQCLLSDSGQVLLESNIKVLPTWSTPVQPMALIVLGGVAGLLLFIGLGIFFCVRCRHRRRQAERMSQIKRLLSEKKTCQCPHRFQKTCSPI

(19) United States
(12) Patent Application Publication
(10) Pub. No.: US 2016/0008462 A1
(43) Pub. Date: Jan. 14, 2016

(54) VETERINARY COMPOSITION AND METHOD OF IMPROVING LIVABILITY OF ANIMALS, PROMOTING LIVE WEIGHT GAIN IN MAMMALS AND BIRDS, ENHANCING THE EFFECTIVENESS OF IMMUNIZATION, AND PREVENTING AND/OR TREATING INFECTIOUS DISEASES (VARIANTS)

(71) Applicant: Oleg Iliich Epshtein, Moscow (RU)

(72) Inventor: Oleg Iliich Epshtein, Moscow (RU)

(21) Appl. No.: 14/730,694

(22) Filed: Jun. 4, 2015

(30) Foreign Application Priority Data
Jun. 6, 2014 (RU) 2014123129

(51) Int. Cl. A61K 39/395

(52) U.S. Cl. CPC A61K 39/3955; A61K 2039/507

(57) ABSTRACT

The present application provides a pharmaceutical composition comprising a) an activated-potentiated form of an antibody to human insulin receptor and b) an activated-potentiated form of an antibody to human interferon gamma, which can be used for improving livability of animals, primarily, promoting live-weight gain and growth of mammals and birds (preferably food-producing animals and poultry), enhancing the effectiveness of immunization, preventing and/or treating a broad range of diseases (including infectious diseases of various etiology), and increasing livestock performance, reproduction and survival.

This application claims priority to Russian Patent Application No. 2014123129, filed Jun. 6, 2014, all of which are incorporated by reference in its entirety.

### FIELD

This invention relates to pharmaceutical composition and methods of improving livability of animals, primarily, promoting live-weight gain and growth of mammals and birds (preferably food-producing animals and poultry), enhancing the effectiveness of immunization, preventing and/or treating a broad range of diseases (including infectious diseases of various etiology), and increasing livestock performance, reproduction and survival.

### BACKGROUND

Over past few decades, the world's meat producing industry has been undergoing brisk, spasmodic changes in search of the ways to meet the growing consumer demand. Alongside with this, scientific interest in organic production of mammalian and poultry meat has increased immensely.

Livestock and poultry breeding industry relies on a wide use of non-nutritional food supplements, primarily antibiotics, in order to improve performance and immune status of animals. Some of these supplements are indicated for chemotherapeutic and prophylactic purposes, whereas others are employed as growth promoters.

Prolonged use of feeds supplemented with subtherapeutic doses of such additives may result in an accumulation of their residuals in animal-derived products and development of drug-resistant microorganisms in humans.

The use of antibiotics as the pivotal part of breeding programmes has recently been abandoned by most poultry and mammalian meat producers. The EU has issued a recommendation against the use of antibiotics, including chlortetracycline, as growth stimulants and means to enhance production efficiency and reduce livestock mortality (Perreten V. 2003 Use of antimicrobials in food producing animals in Switzerland and the European Union (EU). Mitt. Lebensm. Hyg. 94:155-163). This is justified by the fact that the resistance of microorganisms to antibiotics and their fragments in meat products may be detrimental to users' health. The ban on synthetic feed supplements has spawned high-profile research and investigational development of alternative animal health and performance enhancers that could meet the needs of continuously evolving meat industry. The most important selection efforts are focused on growth promotion, though such interferences have been found to negatively correlate with the immune status of animals and poultry. Most investigations are now dealing with the issue of designing new medicines that could be used as growth promoters in the husbandry of both mammals and birds and enhance livestock performance and immunological resistance to numerous diseases. Growth promoters, such as probiotics, prebiotics and immunomodulators, were developed as an alternative to antibiotic growth stimulants. For such agents it has been shown that mammalian and bird species that are genetically characterized by a large body size are able to elicit a far less prominent humoral immune response (Miller L. L., Siegel P. B., and Dunnington E. A. 1992. Inheritance of antibody response to sheep erythrocytes in lines of chickens divergently selected for fifty-six-day body weight and their crosses. Poult. Sci., 71: 47-52).

There are veterinary drug compositions known in the art that are used for the prevention/treatment of a large number of diseases, including infectious ones (RU 20059408 CI, A61K9/08, 1996; RU 2440121 C1, A61K31/7016, 2011).

Also, there is a range of plant-derived food supplements, known in the art, including different microelements, ferments and synthetic compounds (RU 2007456 C1, A23K1/65, 1994; RU 2105496 C1, A23K1/16, 1998; RU 2340204 C1, A23K1/00, 2008; RU 2420089 C1, A23K1/00, 2011; RU 2450532 C1, A23K1/00, 2012), added in large amounts to animal feed rations.

In addition, there are growth promoters, known in the art, used to increase body weight gain in animals (RU 2102063 C1, A23K1/00, 1998; RU 2268043 C2, A23K31/41, 2006; I. F. KLENOVA, N. A. YAREMENKO. Veterinary Drugs in Russia, Guide. Moscow, Sel'khozizdat, 2001, P.171-174; N. V. DEMIDOV. Anthelmintics in Veterinary Practice. Moscow, “Kolos” Publisher, 1982, P.250-298).

However, the abovementioned drugs generally have a limited efficacy range and may cause adverse effects.

The therapeutic effect of an extremely diluted form (or ultra-low form) of antibodies potentized by homeopathic technology (activated-potentiated form) has been discovered by Dr. Oleg I. Epshtein. For example, U.S. Pat. No. 7,582,294 discloses a medicament for treating Benign Prostatic Hyperplasia or prostatitis by administration of a homeopathically activated form of antibodies to prostate specific antigen (PSA). Ultra-low doses of antibodies to gamma interferon have been shown to be useful in the treatment and prophylaxis of diseases of viral etiology. See U.S. Pat. No. 7,572,441, which is incorporated herein by reference in its entirety.

The present invention is directed to an effective and safe pharmaceutical composition for use in and methods of its use for improving livability of animals, primarily, promoting live-weight gain and growth of mammals and birds (preferably food-producing animals and poultry), enhancing the effectiveness of immunization, preventing and/or treating a broad range of diseases (including infectious diseases of various etiology), increasing animal welfare and increasing livestock performance, reproduction and survival. It is specifically contemplated that the pharmaceutical compositions of this invention may be used in human patients as well as for veterinary purposes, although they were so far developed for veterinary use.

The solution to the existing problem is presented in form of a pharmaceutical composition for use in humans, non-human animals or birds comprising a) an activated-potentiated form of an antibody to human insulin receptor and b) an activated-potentiated form of an antibody to human interferon gamma.

### SUMMARY

In one aspect, the invention provides a pharmaceutical composition for use in humans, non-human animals or birds comprising a) an activated-potentiated form of an antibody to human insulin receptor and b) an activated-potentiated form of an antibody to human interferon gamma. Preferably, the invention provides a pharmaceutical composition comprising a) an activated-potentiated form of an antibody a C-terminal fragment of the insulin receptor β-subunit and b) an activated-potentiated form of an antibody to human interferon gamma. In an embodiment, the pharmaceutical composition comprises an activated-potentiated form of an antibody a C-terminal fragment of the insulin receptor β-subunit and an activated-potentiated form of an antibody to human interferon gamma, wherein the activated-potentiated form of an antibody to a C-terminal fragment of the insulin receptor β-subunit and activated-potentiated form of an antibody to human interferon gamma are each represented by an aqueous or aqueous-alcoholic solution with the activity achieved through repeated sequential dilution of the antibody primary matrix solution in a water or alcohol-water solvent, coupled with external mechanical treatment of each dilution.

In one embodiment of the invention, the pharmaceutical composition may be presented as a solid dosage form comprising a technologically required amount of the neutral carrier saturated with the activated-potentiated form of an antibody to a C-terminal fragment of the insulin receptor β-subunit, and the activated-potentiated form of an antibody to human interferon gamma, in combination with pharmaceutically acceptable excipients.

In this embodiment of the pharmaceutical composition, the activated-potentiated form of antibodies to a C-terminal fragment of the insulin receptor β-subunit and activated-potentiated form of an antibody to human interferon gamma may be obtained by repeated sequential dilution of the primary solutions of antibodies to a C-terminal of the insulin receptor β-subunit and to human interferon gamma, coupled with external impact—shaking at the end of each dilution step, with the primary solution concentration of 0.5÷5.0 mg/ml.

It is particularly contemplated that each component is used in the form of a mixture of centesimal dilutions obtained according to a homeopathic manufacturing method, and said pharmaceutically acceptable excipients include lactose, microcrystalline cellulose and magnesium stearate.

In another embodiment of the invention, the pharmaceutically acceptable excipients may include isomalt, sodium cyclamate, sodium saccharine, anhydrous citric acid, and magnesium stearate.

In one aspect, the pharmaceutical composition containing the activated-potentiated form of an antibody to a C-terminal fragment of the insulin receptor β-subunit, and activated-potentiated form of an antibody to human interferon gamma provides a method of improving livability of animals.

In another aspect, the activated-potentiated forms of an antibody to a C-terminal fragment of the insulin receptor β-subunit and antibody to human interferon gamma provide a method of promoting body weight gain in mammals and birds.

Further, the activated-potentiated forms of an antibody to a C-terminal fragment of the insulin receptor β-subunit and antibody to human interferon gamma provide a method of enhancing the effectiveness of immunization in mammals and birds.

In another aspect, the activated-potentiated forms of an antibody to a C-terminal fragment of the insulin receptor β-subunit and antibody to human interferon gamma provide a method of preventing and/or treating infectious diseases of mammals and birds.

The method for improving livability of food-producing animals (mammals and birds) involves administering to an animal an activated-potentiated form of an antibody to the insulin receptor β-subunit, and an activated-potentiated form of an antibody to human interferon gamma.

Particularly contemplated is a variant of this aspect comprising administration of an activated-potentiated form of an antibody to a C-terminal fragment of the insulin receptor β-subunit and activated-potentiated form of an antibody to human interferon gamma, wherein either activated-potentiated form is represented by an aqueous or aqueous-alcoholic solution with the activity achieved through repeated sequential dilution of the primary matrix solutions of the antibodies to a C-terminal fragment of the insulin receptor β-subunit and to human interferon gamma in a water or alcohol-water solvent, coupled with shaking of each dilution.

In accordance with this aspect of the invention, a single preparation—single unit dosage form—incorporates a mixture of various dilutions of antibodies to a C-terminal fragment of the insulin receptor β-subunit and to human interferon gamma obtained according to a homeopathic manufacturing method.

The method of promoting body weight gain in mammals and birds involves administering to an animal an activated-potentiated form of an antibody to the insulin receptor β-subunit, and an activated-potentiated form of an antibody to human interferon gamma.

Particularly contemplated is a variant of this aspect comprising administration of an activated-potentiated form of an antibody to a C-terminal fragment of the insulin receptor β-subunit and activated-potentiated form of an antibody to interferon gamma, wherein either activated-potentiated form is represented by an aqueous or aqueous-alcoholic solution with the activity achieved through repeated sequential dilution of the primary (matrix) solutions of the antibodies to a C-terminal fragment of the insulin receptor β-subunit and to human interferon gamma in a water or alcohol-water solvent, coupled with external mechanical treatment—shaking of each dilution.

It is particularly contemplated that a single preparation—single unit dosage form—incorporates a mixture of various dilutions of antibodies to a C-terminal fragment of the insulin receptor β-subunit and to human interferon gamma obtained according to a homeopathic manufacturing method.

The method of enhancing the effectiveness of immunization in mammals and birds involves administering to an animal an activated-potentiated form of an antibody to the insulin receptor β-subunit, and an activated-potentiated form of an antibody to human interferon gamma.

Particularly contemplated is a variant of this aspect comprising administration of an activated-potentiated form of an antibody to a C-terminal fragment of the insulin receptor β-subunit and activated-potentiated form of an antibody to interferon gamma, wherein either activated-potentiated form is represented by an aqueous or aqueous-alcoholic solution with the activity achieved through repeated sequential dilution of the primary (matrix) solutions of the antibodies to a C-terminal fragment of the insulin receptor β-subunit and to human interferon gamma in a water or alcohol-water solvent, coupled with external mechanical treatment—shaking of each dilution.

It is particularly contemplated that a single preparation—single unit dosage form—incorporates a mixture of various dilutions of antibodies to a C-terminal fragment of the insulin receptor β-subunit and to human interferon gamma obtained according to a homeopathic manufacturing method.

The method of preventing and/or treating infectious diseases of mammals and birds involves administering to an animal an activated-potentiated form of an antibody to the insulin receptor β-subunit, and an activated-potentiated form of an antibody to human interferon gamma.

Particularly contemplated is a variant of this aspect comprising administration of an activated-potentiated form of an antibody to a C-terminal fragment of the insulin receptor β-subunit and activated-potentiated form of an antibody to interferon gamma, wherein either activated-potentiated form is represented by an aqueous or aqueous-alcoholic solution with the activity achieved through repeated sequential dilution of the primary (matrix) solutions of the antibodies to a C-terminal fragment of the insulin receptor β-subunit and to human interferon gamma in a water or alcohol-water solvent, coupled with external mechanical treatment—shaking of each dilution.

It is particularly contemplated that a single preparation—single unit dosage form—incorporates a mixture of various dilutions of antibodies to a C-terminal fragment of the insulin receptor β-subunit and to human interferon gamma obtained according to a homeopathic manufacturing method.

In one variant of the invention, the pharmaceutical composition additionally comprises an activated-potentiated form of an antibody CD4 receptor, wherein the activated-potentiated form of an antibody to a C-terminal fragment of to insulin receptor β-subunit, activated-potentiated form of an antibody to human interferon gamma, and activated-potentiated form of an antibody to CD4 receptor are each represented by an activated-potentiated aqueous or aqueous-alcoholic solution with the activity achieved through repeated sequential dilution of the antibody primary (matrix) solution in a water or alcohol-water solvent, coupled with external mechanical treatment—shaking of each dilution.

In one embodiment of the invention, the pharmaceutical composition may be presented as a compound preparation in a solid dosage form comprising a technologically required amount of the neutral carrier saturated with the activated-potentiated form of an antibody to a C-terminal fragment of the insulin receptor β-subunit, activated-potentiated form of an antibody to human interferon gamma and activated-potentiated form an antibody to CD4 receptor, in combination with pharmaceutically acceptable excipients.

In this embodiment of the pharmaceutical composition, said activated-potentiated form of antibodies to a C-terminal fragment of the insulin receptor β-subunit, activated-potentiated form of an antibody to human interferon gamma and activated-potentiated form an antibody to CD4 receptor are obtained by repeated sequential dilution of the primary solutions of antibodies to a C-terminal of the insulin receptor β-subunit, to human interferon gamma, and to CD4, coupled with external impact—shaking at the end of each dilution step, with the primary solution concentration of 0.5÷ 5.0 mg/ml.

It is particularly contemplated that each of the components is used in the form of a mixture of various, primarily centesimal, dilutions obtained according to a homeopathic manufacturing method, and said pharmaceutically acceptable excipients include lactose, microcrystalline cellulose and magnesium stearate.

Additionally, the pharmaceutically acceptable additives incorporate isomalt, sodium cyclamate, sodium saccharine, anhydrous citric acid, and magnesium stearate.

In one aspect, the pharmaceutical composition containing the activated-potentiated form of an antibody to a C-terminal fragment of the insulin receptor β-subunit, activated-potentiated form of an antibody to human interferon gamma and activated-potentiated form of an antibody to CD4 receptor provides a method of improving livability of animals.

In another aspect, the pharmaceutical composition provides a method of promoting body weight gain in mammals and birds.

In another aspect, the pharmaceutical composition provides a method of enhancing the effectiveness of immunization in mammals and birds.

Further, said pharmaceutical composition provides a method of preventing and/or treating infectious diseases of mammals and birds.

The method of improving livability of animals (mammals and birds) involves administering to an animal an activated-potentiated form of an antibody to the insulin receptor β-subunit, activated-potentiated form of an antibody to human interferon gamma and an activated-potentiated form of an antibody to CD4 receptor.

Particularly contemplated is a variant of this aspect comprising administration of an activated-potentiated form of an antibody to a C-terminal fragment of the insulin receptor β-subunit, activated-potentiated form of an antibody to interferon gamma and an an activated-potentiated form of an antibody to CD4 receptor, wherein each activated-potentiated form is represented by an aqueous or aqueous-alcoholic solution with the activity achieved through repeated sequential dilution of the primary (matrix) solutions of the antibodies to a C-terminal fragment of the insulin receptor β-subunit, to human interferon gamma, and to CD4 in a water or alcohol-water solvent, coupled with external mechanical treatment—shaking of each dilution.

It is particularly contemplated that a single preparation—single unit dosage form—incorporates a mixture of various dilutions of antibodies to a C-terminal fragment of the insulin receptor β-subunit, to human interferon gamma, and to CD4 obtained according to a homeopathic manufacturing method.

The method of promoting body weight gain in mammals and birds involves administering to an animal an activated-potentiated form of an antibody to the insulin receptor β-subunit, activated-potentiated form of an antibody to human interferon gamma and an activated-potentiated form of an antibody to CD4 receptor.

Particularly contemplated is a variant of this aspect comprising administration of an activated-potentiated form of an antibody to a C-terminal fragment of the insulin receptor β-subunit, activated-potentiated form of an antibody to interferon gamma and an an activated-potentiated form of an antibody to CD4 receptor, wherein each activated-potentiated form is represented by an aqueous or aqueous-alcoholic solution with the activity achieved through repeated sequential dilution of the primary (matrix) solutions of the antibodies to a C-terminal fragment of the insulin receptor β-subunit, to human interferon gamma, and to CD4 in a water or alcohol-water solvent, coupled with external mechanical treatment—shaking of each dilution.

In accordance with this aspect, a single preparation—single unit dosage form—incorporates a mixture of various dilutions of antibodies to a C-terminal fragment of the insulin receptor β-subunit, to human interferon gamma, and to CD4 obtained according to a homeopathic manufacturing method.

The method enhancing the effectiveness of immunization in mammals and birds involves administering to an animal an activated-potentiated form of an antibody to the insulin receptor β-subunit, activated-potentiated form of an antibody to human interferon gamma and an activated-potentiated form of an antibody to CD4 receptor.

Particularly contemplated is a variant of this aspect comprising administration of an activated-potentiated form of an antibody to a C-terminal fragment of the insulin receptor β-subunit, activated-potentiated form of an antibody to interferon gamma and an an activated-potentiated form of an antibody to CD4 receptor, wherein each activated-potentiated form is represented by an aqueous or aqueous-alcoholic solution with the activity achieved through repeated sequential dilution of the primary (matrix) solutions of the antibodies to a C-terminal fragment of the insulin receptor β-subunit, to human interferon gamma, and to CD4 in a water or alcohol-water solvent, coupled with external mechanical treatment—shaking of each dilution.

It is particularly contemplated that a single preparation—single unit dosage form—incorporates a mixture of various dilutions of antibodies to a C-terminal fragment of the insulin receptor β-subunit, to human interferon gamma, and to CD4 obtained according to a homeopathic manufacturing method.

The method of preventing and/or treating infectious diseases of mammals and birds involves administering to an animal an activated-potentiated form of an antibody to the insulin receptor β-subunit, activated-potentiated form of an antibody to human interferon gamma and an activated-potentiated form of an antibody to CD4 receptor.

Particularly contemplated is a variant of this aspect comprising administration of an activated-potentiated form of an antibody to a C-terminal fragment of the insulin receptor β-subunit, activated-potentiated form of an antibody to interferon gamma and an an activated-potentiated form of an antibody to CD4 receptor, wherein each activated-potentiated form is represented by an aqueous or aqueous-alcoholic solution with the activity achieved through repeated sequential dilution of the primary (matrix) solutions of the antibodies to a C-terminal fragment of the insulin receptor β-subunit, to human interferon gamma, and to CD4 in a water or alcohol-water solvent, coupled with external mechanical treatment—shaking of each dilution.

In accordance with this aspect, a single preparation—single unit dosage form—incorporates a mixture of various dilutions of antibodies to a C-terminal fragment of the insulin receptor β-subunit, to human interferon gamma, and to CD4 obtained according to a homeopathic manufacturing method.

In accordance with the invention, the maximum beneficial effect on the livability of food-producing animals, mammals and birds may be achieved through regular, long-term administration of the veterinary composition.

As a method of promoting body weight gain in mammals and birds, the veterinary composition is administered throughout the fattening period, from the first to the last day of life.

For the purposes of increasing stock performance and survival, preventing infectious diseases and enhancing the effectiveness of immunization, the veterinary composition is preferably administered for a total of three/four 4-7-day periods.

In accordance with the invention, the claimed aqueous or aqueous-alcoholic solutions have pronounced activity (potency) acquired during the treatment process involving sequential decrease in the concentration of the initial substance—antibodies to the insulin receptor β-subunit (C-terminal fragment of insulin receptor β-subunit), to human interferon gamma, and to CD4 receptor, said activity attributed to the ability of the activated-potentiated form of an antibody to the insulin receptor β-subunit (C-terminal fragment of insulin receptor β-subunit) to promote the cellular metabolic processes, interfering with the carbohydrate metabolism and resulting in increased rates of body weight gain with reduced feed consumption. Through its effect on various immune mediators, the activated-potentiated form of an antibody to human interferon gamma enhances the key natural resistance parameters of the body, influences the endogenous interferon system, enhances humoral and cellular immune responses and functional activity of phagocytes and natural killer cells (NK cells), and exhibits antiviral action against DNA- and RNA-viruses, in particular influenza viruses (H3N2, H3N8, H1N1), including avian influenza virus, herpes simplex virus type 2, human immunodeficiency virus (HIV-1), and feline immunodeficiency virus (FIV). The activated-potentiated form of an antibody to human interferon gamma has antibacterial effects when used as part of combination therapy for bacterial infections and prevention of bacterial complications.

The activated-potentiated form of an antibody to CD4 receptor regulates the CD4 receptor functioning, increasing thereby the functional activity of CD4 lymphocytes and normalizing CD4/CD8 immunoregulatory index and distribution of immunocompetent cell subpopulations (CD3, CD4, CD8, CD16, CD20).

The combined use of the claimed components of the veterinary composition allows for higher activity of its constituents, and, as a result, effective improvement of animals' livability.

The above mentioned are presumably the mechanisms through which the veterinary composition, in its various variants and embodiments, regulates metabolic processes and exerts antiviral and antibacterial action. The compound has efficacy against avian infections caused by viral, bacterial and mycoplasmal agents, Newcastle disease and Gumboro disease; and is effective in improving immunity, enhancing the humoral response to vaccines and thereby increasing, the effectiveness of immunization, livestock survival and performance with reduced rates of feed consumption, which has been demonstrated experimentally.

In the proposed aspects of use, the activated-potentiated form of an antibody to the insulin receptor β-subunit (C-terminal fragment of insulin receptor β-subunit), including the combination with the activated-potentiated forms of an antibody to human interferon gamma and antibody to CD4 receptor, broadens the range of compounds for improving animals' livability, promoting body weight gain in mammals and birds, enhancing the effectiveness of immunization, and preventing and/or treating infectious diseases, with high survival rate provided in mammals and birds. In said aspects of use, the invention produces neither adverse effects nor general toxicity or immunotoxicity effects, causes no local irritation or allergic sensitization and has no reproductive toxicity (which is attributed to the virtual absence of or ultra-low molecular concentration of the highly diluted initial substance). A long-term administration of the veterinary composition is not associated with adverse events such as hypoglycemia or acidosis. Particularly contemplated is administration of the claimed veterinary composition in combination with other bioactive feed supplements and/or drug products used both for promoting body weight gain and growth of food-producing animals, enhancing the effectiveness of immunization, and treating and/or preventing infectious diseases.

### DESCRIPTION OF THE FIGURES

FIG. 1—Illustrates feed intake values in the breeding units.

FIG. 2—Illustrates the effect of tested preparations on carcass quality distribution of broilers (by categories).

### DETAILED DESCRIPTION

The invention is defined with reference to the appended claims. With respect to the claims, the glossary that follows provides the relevant definitions.

The term “antibody” as used herein shall mean an immunoglobulin that specifically binds to, and is thereby defined as complementary with, a particular spatial and polar organization of another molecule. Antibodies as recited in the claims may include a complete immunoglobulin or fragment thereof, may be natural, polyclonal or monoclonal, and may include various classes and isotypes, such as IgA, IgD, IgE, IgG1, IgG2a, IgG2b and IgG3, IgM, etc. Fragments thereof may include Fab, Fv and F(ab′)2, Fab′, and the like. The singular “antibody” includes plural “antibodies.”

The term “activated-potentiated form” or “potentiated form” respectively, with respect to antibodies recited herein is used to denote a product of homeopathic potentization of any initial solution of antibodies. “Homeopathic potentization” denotes the use of methods of homeopathy to impart homeopathic potency to an initial solution of relevant substance. Although not so limited, ‘homeopathic potentization” may involve, for example, repeated consecutive dilutions combined with external treatment, particularly (mechanical) shaking. In other words, an initial solution of antibody is subjected to consecutive repeated dilution and multiple vertical shaking of each obtained solution in accordance with homeopathic technology. The preferred concentration of the initial solution of antibody in the solvent, preferably water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³⁰ and 100²⁰⁰ times, respectively, which is equivalent to centesimal homeopathic dilutions (C12, C30, and C200) or the use of the mixture of three aqueous or aqueous-alcohol dilutions of the primary matrix solution of antibodies diluted 100¹², 100³⁰ and 100⁵⁰ times, respectively, which is equivalent to centesimal homeopathic dilutions (C12, C30 and C50). Examples of homeopathic potentization are described in U.S. Pat. Nos. 7,572,441 and 7,582,294, which are incorporated herein by reference in their entirety and for the purpose stated. While the term “activated-potentiated form” is used in the claims, the term “ultra-low doses” is used in the examples. The term “ultra-low doses” became a term of art in the field of art created by study and use of homeopathically diluted and potentized form of substance. The term “ultra-low dose” or “ultra-low doses” is meant as fully supportive and primarily synonymous with the term ‘activated-potentiated” form used in the claims.

In other words, an antibody is in the “activated-potentiated” form when three factors are present. First, the “activated-potentiated” form of the antibody is a product of a preparation process well accepted in the homeopathic art. Second, the “activated-potentiated” form of antibody must have biological activity determined by methods well accepted in modern pharmacology. And third, the biological activity exhibited by the “activated potentiated” form of the antibody cannot be explained by the presence of the molecular form of the antibody in the final product of the homeopathic process.

For example, the activated potentiated form of antibodies may be prepared by subjecting an initial, isolated antibody in a molecular form to consecutive multiple dilutions coupled with an external impact, such as mechanical shaking. The external treatment in the course of concentration reduction may also be accomplished, for example, by exposure to ultrasonic, electromagnetic, or other physical factors. V. Schwabe “Homeopathic medicines”, M., 1967, U.S. Pat. Nos. 7,229,648 and 4,311,897, which are incorporated by reference in their entirety and for the purpose stated, describe such processes that are well accepted methods of homeopathic potentiation in the homeopathic art. This procedure gives rise to a uniform decrease in molecular concentration of the initial molecular form of the antibody. This procedure is repeated until the desired homeopathic potency is obtained. For the individual antibody, the required homeopathic potency can be determined by subjecting the intermediate dilutions to biological testing in the desired pharmacological model. Although not so limited, ‘homeopathic potentization” may involve, for example, repeated consecutive dilutions combined with external treatment, particularly vertical mechanical shaking. In other words, an initial solution of antibody is subjected to consecutive repeated dilution and multiple vertical shaking of each obtained solution in accordance with homeopathic technology. The preferred concentration of the initial solution of antibody in the solvent, preferably, water or a water-ethyl alcohol mixture, ranges from about 0.5 to about 5.0 mg/ml. The preferred procedure for preparing each component, i.e. antibody solution, is the use of the mixture of three aqueous or aqueous-alcohol dilutions of the primary matrix solution (mother tincture) of antibodies diluted 100¹², 100³⁰ and 100²⁰⁰ times, respectively, which is equivalent to centesimal homeopathic dilutions C12, C30 and C200 or the mixture of three aqueous or aqueous-alcohol dilutions of the primary matrix solution (mother tincture) of antibodies diluted 100¹², 100³⁰ and 100⁵⁰ times, respectively, which is equivalent to centesimal homeopathic dilutions C12, C30 and C50. Examples of how to obtain the desired potency are also provided, for example, in U.S. Pat. Nos. 7,229,648 and 4,311,897, which are incorporated by reference for the purpose stated. The procedure applicable to the “activated potentiated” form of the antibodies described herein is described in more detail below.

There has been a considerable amount of controversy regarding homeopathic treatment. While the present invention relies on accepted homeopathic processes to obtain the “activated-potentiated” form of antibodies, it does not rely solely on homeopathy in human subjects for evidence of activity. It has been surprisingly discovered by the inventor of the present application and amply demonstrated in the accepted pharmacological models that the solvent ultimately obtained from consecutive multiple dilution of a starting molecular form of an antibody has definitive activity unrelated to the presence of the traces of the molecular form of the antibody in the target dilution. The “activated-potentiated” form of the antibody provided herein are tested for biological activity in well accepted pharmacological models of activity, either in appropriate in vitro experiments, or in vivo in suitable animal models. The experiments provided further below provide evidence of biological activity in such models.

Also, the claimed “activated-potentiated” form of antibody encompass only solutions or solid preparations the biological activity of which cannot be explained by the presence of the molecular form of the antibody remaining from the initial, starting solution. In other words, while it is contemplated that the “activated-potentiated” form of the antibody may contain traces of the initial molecular form of the antibody, one skilled in the art could not attribute the observed biological activity in the accepted pharmacological models to the remaining molecular form of the antibody with any degree of plausibility due to the extremely low concentrations of the molecular form of the antibody remaining after the consecutive dilutions. While the invention is not limited by any specific theory, the biological activity of the “activated-potentiated” form of the antibodies of the present invention is not attributable to the initial molecular form of the antibody. Preferred is the “activated-potentiated” form of antibody in liquid or solid form in which the concentration of the initial molecular form of the antibody is below the limit of detection of the accepted analytical techniques, such as capillary electrophoresis and High Performance Liquid Chromatography. Particularly preferred is the “activated-potentiated” form of antibody in liquid or solid form in which the concentration of the initial molecular form of the antibody is below the Avogadro number. In pharmacology of molecular forms of therapeutic substances, it is common practice to create a dose-response curve in which the level of pharmacological response is plotted against the concentration of the active drug administered to the subject or tested in vitro. The minimal level of the drug which produces any detectable response is known as a threshold dose. It is specifically contemplated and preferred that the “activated-potentiated” form of the antibodies contains molecular antibody, if any, at a concentration below the threshold dose for the molecular form of the antibody in the given biological model.

The present invention provides a veterinary composition for improving livability of animals, primarily, promoting live-weight gain and growth of mammals and birds (preferably food-producing animals and poultry), enhancing the effectiveness of immunization, preventing and/or treating a broad range of diseases (including infectious diseases of various etiology), and increasing livestock performance, reproduction and survival.

The pharmaceutical composition in accordance with this aspect of the invention may be in the liquid form or in solid form. Each of the activated potentiated forms of the antibodies included in the pharmaceutical composition is prepared from an initial molecular form of the antibody via a process accepted in homeopathic art. The starting antibodies may be monoclonal, or polyclonal antibodies prepared in accordance with known processes, for example, as described in Immunotechniques, G. Frimel, M., “Meditsyna”, 1987, p. 9-33; “Hum. Antibodies. Monoclonal and recombinant antibodies, 30 years after” by Laffly E., Sodoyer R. —2005—Vol. 14. —N 1-2. P.33-55, both incorporated herein by reference.

Monoclonal antibodies may be obtained, e.g., by means of hybridoma technology. The initial stage of the process includes immunization based on the principles already developed in course of polyclonal antisera preparation. Further stages of work involve production of hybrid cells generating clones of antibodies with identical specificity. Their separate isolation is performed using the same methods as in case of polyclonal antisera preparation.

Polyclonal antibodies may be obtained via active immunization of animals. For this purpose, for example, suitable animals (e.g. rabbits) receive a series of injections of the appropriate antigen (insulin receptor, interferon gamma or CD4). The animals' immune system generates corresponding antibodies, which are collected from the animals in a known manner. This procedure enables preparation of a monospecific antibody-rich serum. If desired, the serum containing antibodies may be purified, e.g., using affine chromatography, fractionation by salt precipitation, or ion-exchange chromatography. The resulting purified, antibody-enriched serum may be used as a starting material for preparation of the activated-potentiated form of the antibodies. The preferred concentration of the resulting initial solution of antibody in the solvent, preferably, water or water-ethyl alcohol mixture, ranges from about 0.5 to about 5.0 mg/ml.

The preferred procedure for preparing each component is the use of the mixture of three aqueous-alcohol dilutions of the primary matrix solution of antibodies diluted 100¹², 100³⁰ and 100²⁰⁰ times, respectively, which is equivalent to centesimal homeopathic dilutions C12, C30 and C200. To prepare a solid dosage form, a solid carrier is treated with the desired dilution obtained via the homeopathic process. To obtain a solid unit dosage form of the combination of the invention, the carrier mass is impregnated with each of the dilutions. Both orders of impregnation are suitable to prepare the desired combination dosage form.

In the preferred embodiment, the starting material for the preparation of the activated potentiated form that comprise the combination of the invention is polyclonal, animal-raised antibody to the corresponding antigen, namely, C-terminal fragment of beta subunit of human insulin receptor or insulin receptor, interferon gamma and CD4. To obtain the activated-potentiated form of polyclonal antibodies to C-terminal fragment of beta subunit of human insulin receptor, the desired antigen may be injected as immunogen into a laboratory animal, preferably, rabbits′. Peptides of particular interest may include at least about 3 amino acids, usually at least about 10 on either side of the sequence, preferably having at least 3 amino acids at the C-terminal side. The following sequences of human insulin receptor are specifically contemplated as suitable antigens:

Entire alpha-subunit of human insulin receptor:

```
SEQ ID NO: 1
His Leu Tyr
  28 30
Pro Gly Glu Val Cys Pro Gly Met Asp Ile Arg Asn Asn Leu Thr
  31 35 40 45
Arg Leu His Glu Leu Glu Asn Cys Ser Val Ile Glu Gly His Leu
  46 50 55 60
Gln Ile Leu Leu Met Phe Lys Thr Arg Pro Glu Asp Phe Arg Asp
61 65 70 75
Leu Ser Phe Pro Lys Leu Ile Met Ile Thr Asp Tyr Leu Leu Leu
76 80 85 90
Phe Arg Val Tyr Gly Leu Glu Ser Leu Lys Asp Leu Phe Pro Asn
91 95 100 105
Leu Thr Val Ile Arg Gly Ser Arg Leu Phe Phe Asn Tyr Ala Leu
106 110 115 120
Val Ile Phe Glu Met Val His Leu Lys Glu Leu Gly Leu Tyr Asn
121 125 130 135
Leu Met Asn Ile Thr Arg Gly Ser Val Arg Ile Glu Lys Asn Asn
136 140 145 150
Glu Leu Cys Tyr Leu Ala Thr Ile Asp Trp Ser Arg Ile Leu Asp
151 155 160 165
Ser Val Glu Asp Asn Tyr Ile Val Leu Asn Lys Asp Asp Asn Glu
166 170 175 180
Glu Cys Gly Asp Ile Cys Pro Gly Thr Ala Lys Gly Lys Thr Asn
181 185 190 195
Cys Pro Ala Thr Val Ile Asn Gly Gln Phe Val Glu Arg Cys Trp
196 200 205 210
Thr His Ser His Cys Gln Lys Val Cys Pro Thr Ile Cys Lys Ser
211 215 220 225
His Gly Cys Thr Ala Glu Gly Leu Cys Cys His Ser Glu Cys Leu
226 230 235 240
Gly Asn Cys Ser Gln Pro Asp Asp Pro Thr Lys Cys Val Ala Cys
241 245 250 255
Arg Asn Phe Tyr Leu Asp Gly Arg Cys Val Glu Thr Cys Pro Pro
256 260 265 270
Pro Tyr Tyr His Phe Gln Asp Trp Arg Cys Val Asn Phe Ser Phe
271 275 280 285
Cys Gln Asp Leu His His Lys Cys Lys Asn Ser Arg Arg Gln Gly
286 290 295 300
Cys His Gln Tyr Val Ile His Asn Asn Lys Cys Ile Pro Glu Cys
301 305 310 315
Pro Ser Gly Tyr Thr Met Asn Ser Ser Asn Leu Leu Cys Thr Pro
316 320 325 330
Cys Leu Gly Pro Cys Pro Lys Val Cys His Leu Leu Glu Gly Glu
331 335 340 345
Lys Thr Ile Asp Ser Val Thr Ser Ala Gln Glu Leu Arg Gly Cys
346 350 355 360
Thr Val Ile Asn Gly Ser Leu Ile Ile Asn Ile Arg Gly Gly Asn
361 365 370 375
Asn Leu Ala Ala Glu Leu Glu Ala Asn Leu Gly Leu Ile Glu Glu
376 380 385 390
Ile Ser Gly Tyr Leu Lys Ile Arg Arg Ser Tyr Ala Leu Val Ser
391 395 400 405
Leu Ser Phe Phe Arg Lys Leu Arg Leu Ile Arg Gly Glu Thr Leu
406 410 415 420
Glu Ile Gly Asn Tyr Ser Phe Tyr Ala Leu Asp Asn Gln Asn Leu
421 425 430 435
Arg Gln Leu Trp Asp Trp Ser Lys His Asn Leu Thr Ile Thr Gln
436 440 445 450
Gly Lys Leu Phe Phe His Tyr Asn Pro Lys Leu Cys Leu Ser Glu
451 455 460 465
Ile His Lys Met Glu Glu Val Ser Gly Thr Lys Gly Arg Gln Glu
466 470 475 480
Arg Asn Asp Ile Ala Leu Lys Thr Asn Gly Asp Gln Ala Ser Cys
481 485 490 495
Glu Asn Glu Leu Leu Lys Phe Ser Tyr Ile Arg Thr Ser Phe Asp
496 500 505 510
Lys Ile Leu Leu Arg Trp Glu Pro Tyr Trp Pro Pro Asp Phe Arg
511 515 510 525
Asp Leu Leu Gly Phe Met Leu Phe Tyr Lys Glu Ala Pro Tyr Gln
526 530 535 540
Asn Val Thr Glu Phe Asp Gly Gln Asp Ala Cys Gly Ser Asn Ser
541 545 550 555
Trp Thr Val Val Asp Ile Asp Pro Pro Leu Arg Ser Asn Asp Pro
556 560 565 570
Lys Ser Gln Asn His Pro Gly Trp Leu Met Arg Gly Leu Lys Pro
571 575 580 585
Trp Thr Gln Tyr Ala Ile Phe Val Lys Thr Leu Val Thr Phe Ser
586 590 595 600
Asp Glu Arg Arg Thr Tyr Gly Ala Lys Ser Asp Ile Ile Tyr Val
601 605 610 615
Gln Thr Asp Ala Thr Asn Pro Ser Val Pro Leu Asp Pro Ile Ser
616 620 625 630
Val Ser Asn Ser Ser Ser Gln Ile Ile Leu Lys Trp Lys Pro Pro
631 635 640 645
Ser Asp Pro Asn Gly Asn Ile Thr His Tyr Leu Val Phe Trp Glu
646 650 655 660
Arg Gln Ala Glu Asp Ser Glu Leu Phe Glu Leu Asp Tyr Cys Leu
661 665 670 675
Lys Gly Leu Lys Leu Pro Ser Arg Thr Trp Ser Pro Pro Phe Glu
676 680 685 690
Ser Glu Asp Ser Gln Lys His Asn Gln Ser Glu Tyr Glu Asp Ser
691 695 700 705
Ala Gly Glu Cys Cys Ser Cys Pro Lys Thr Asp Ser Gln Ile Leu
706 710 715 720
Lys Glu Leu Glu Glu Ser Ser Phe Arg Lys Thr Phe Glu Asp Tyr
721 725 730 735
Leu His Asn Val Val Phe Val Pro Arg Lys Thr Ser Ser Gly Thr
736 740 745 750
Gly Ala Glu Asp Pro Arg Pro Ser Arg Lys Arg Arg
751 755 760 762
```

### Fragments of Alpha-Subunit of Human Insulin Receptor:

SEQ ID NO: 2
Leu Gly Leu Tyr Asn
131 135
Leu Met Asn Ile Thr Arg Gly Ser Val
136 140 144
SEQ ID NO: 3
Lys Gly Lys Thr Asn
191 195
Cys Pro Ala Thr Val Ile Asn Gly
196 200 203
SEQ ID NO: 4
Trp Ser Lys His Asn Leu Thr Ile Thr Gln
441 445 450
Gly Lys Leu
451 453
SEQ ID NO: 5
Asn Val Thr Glu Phe Asp Gly Gln Asp Ala Cys Gly Ser Asn Ser
541 545 550 555
Trp Thr Val Val Asp
556 560
SEQ ID NO: 6
Asp Ile Ile Tyr Val
611 615
Gln Thr Asp Ala Thr
616 620
SEQ ID NO: 7
Tyr Glu Asp Ser
702 705
Ala Gly Glu Cys Cys Ser Cys Pro Lys Thr Asp Ser Gln Ile
706 710 715 719

### Entire Beta Subunit of Human Insulin Receptor:

SEQ ID NO: 8
Ser Leu Gly
763 765
Asp Val Gly Asn Val Thr Val Ala Val Pro Thr Val Ala Ala Phe
766 770 775 780
Pro Asn Thr Ser Ser Thr Ser Val Pro Thr Ser Pro Glu Glu His
781 785 790 795
Arg Pro Phe Glu Lys Val Val Asn Lys Glu Ser Leu Val Ile Ser
796 800 805 810
Gly Leu Arg His Phe Thr Gly Tyr Arg Ile Glu Leu Gln Ala Cys
811 815 820 825
Asn Gln Asp Thr Pro Glu Glu Arg Cys Ser Val Ala Ala Tyr Val
826 830 835 840
Ser Ala Arg Thr Met Pro Glu Ala Lys Ala Asp Asp Ile Val Gly
841 845 850 855
Pro Val Thr His Glu Ile Phe Glu Asn Asn Val Val His Leu Met
856 860 865 870
Trp Gln Glu Pro Lys Glu Pro Asn Gly Leu Ile Val Leu Tyr Glu
871 875 880 885
Val Ser Tyr Arg Arg Tyr Gly Asp Glu Glu Leu His Leu Cys Val
886 890 895 900
Ser Arg Lys His Phe Ala Leu Glu Arg Gly Cys Arg Leu Arg Gly
901 905 910 915
Leu Ser Pro Gly Asn Tyr Ser Val Arg Ile Arg Ala Thr Ser Leu
916 920 925 930
Ala Gly Asn Gly Ser Trp Thr Glu Pro Thr Tyr Phe Tyr Val Thr
931 935 940 945
Asp Tyr Leu Asp Val Pro Ser Asn Ile Ala Lys Ile Ile Ile Gly
946 950 955 960
Pro Leu Ile Phe Val Phe Leu Phe Ser Val Val Ile Gly Ser Ile
961 965 970 975
Tyr Leu Phe Leu Arg Lys Arg Gln Pro Asp Gly Pro Leu Gly Pro
976 980 985 990
Leu Tyr Ala Ser Ser Asn Pro Glu Tyr Leu Ser Ala Ser Asp Val
991 995 1000 1005
Phe Pro Cys Ser Val Tyr Val Pro Asp Glu Trp Glu Val Ser Arg
1006 1010 1015 1020
Glu Lys Ile Thr Leu Leu Arg Glu Leu Gly Gln Gly Ser Phe Gly
1021 1025 1030 1035
Met Val Tyr Glu Gly Asn Ala Arg Asp Ile Ile Lys Gly Glu Ala
1036 1140 1145 1050
Glu Thr Arg Val Ala Val Lys Thr Val Asn Glu Ser Ala Ser Leu
1051 1155 1160 1065
Arg Glu Arg Ile Glu Phe Leu Asn Glu Ala Ser Val Met Lys Gly
1066 1170 1175 1080
Phe Thr Cys His His Val Val Arg Leu Leu Gly Val Val Ser Lys
1081 1185 1190 1095
Gly Gln Pro Thr Leu Val Val Met Glu Leu Met Ala His Gly Asp
1096 1100 1105 1110
Leu Lys Ser Tyr Leu Arg Ser Leu Arg Pro Glu Ala Glu Asn Asn
1111 1115 1120 1125
Pro Gly Arg Pro Pro Pro Thr Leu Gln Glu Met Ile Gln Met Ala
1126 1130 1135 1140
Ala Glu Ile Ala Asp Gly Met Ala Tyr Leu Asn Ala Lys Lys Phe
1141 1145 1150 1155
Val His Arg Asp Leu Ala Ala Arg Asn Cys Met Val Ala His Asp
1156 1160 1165 1170
Phe Thr Val Lys Ile Gly Asp Phe Gly Met Thr Arg Asp Ile Tyr
1171 1175 1180 1185
Glu Thr Asp Tyr Tyr Arg Lys Gly Gly Lys Gly Leu Leu Pro Val
1186 1190 1195 1200
Arg Trp Met Ala Pro Glu Ser Leu Lys Asp Gly Val Phe Thr Thr
1201 1205 1210 1215
Ser Ser Asp Met Trp Ser Phe Gly Val Val Leu Trp Glu Ile Thr
1216 1220 1225 1230
Ser Leu Ala Glu Gln Pro Tyr Gln Gly Leu Ser Asn Glu Gln Val
1231 1235 1240 1245
Leu Lys Phe Val Met Asp Gly Gly Tyr Leu Asp Gln Pro Asp Asn
1246 1250 1255 1260
Cys Pro Glu Arg Val Thr Asp Leu Met Arg Met Cys Trp Gln Phe
1261 1265 1270 1275
Asn Pro Lys Met Arg Pro Thr Phe Leu Glu Ile Val Asn Leu Leu
1276 1280 1285 1290
Lys Asp Asp Leu His Pro Ser Phe Pro Glu Val Ser Phe Phe His
1291 1295 1300 1305
Ser Glu Glu Asn Lys Ala Pro Glu Ser Glu Glu Leu Glu Met Glu
1306 1310 1315 1320
Phe Glu Asp Met Glu Asn Val Pro Leu Asp Arg Ser Ser His Cys
1321 1325 1330 1335
Gln Arg Glu Glu Ala Gly Gly Arg Asp Gly Gly Ser Ser Leu Gly
1336 1340 1345 1350
Phe Lys Arg Ser Tyr Glu Glu His Ile Pro Tyr Thr His Met Asn
1351 1355 1360 1365
Gly Gly Lys Lys Asn Gly Arg Ile Leu Thr Leu Pro Arg Ser Asn
1366 1370 1375 1380
Pro Ser
13811382

### Fragments of C-Terminal Fragment of Beta Subunit of Human Insulin Receptor:

SEQ ID NO: 9
Lys Lys Asn Gly Arg Ile Leu Thr Leu Pro
1368 1370 1375 1377
SEQ ID NO: 10
Arg Ile Leu Thr Leu Pro Arg Ser Asn
1372 1375 1380
Pro Ser
13811382
SEQ ID NO: 11
Lys Asn Gly Arg Ile Leu Thr
13691370 1375
SEQ ID NO: 12
Gly Gly Lys Lys Asn Gly Arg Ile Leu Thr Leu Pro Arg Ser Asn
1366 1370 1375 1380
Pro Ser
13811382
SEQ ID NO: 13
Asn
1365
Gly Gly Lys Lys Asn Gly Arg Ile Leu Thr Leu Pro Arg Ser Asn
1366 1370 1375 1380
Pro Ser
13811382

The use of human insulin receptor as antigen is also contemplated. The suitable sequence for such antigen is as follow:

```
SEQ ID NO: 14
Met Ala Thr Gly Gly Arg Arg Gly Ala Ala Ala Ala Pro Leu Leu
  1 5 10 15
Val Ala Val Ala Ala Leu Leu Leu Gly Ala Ala Gly His Leu Tyr
  16 20 25 30
Pro Gly Glu Val Cys Pro Gly Met Asp Ile Arg Asn Asn Leu Thr
  31 35 40 45
Arg Leu His Glu Leu Glu Asn Cys Ser Val Ile Glu Gly His Leu
  46 50 55 60
Gln Ile Leu Leu Met Phe Lys Thr Arg Pro Glu Asp Phe Arg Asp
61 65 70 75
Leu Ser Phe Pro Lys Leu Ile Met Ile Thr Asp Tyr Leu Leu Leu
76 80 85 90
Phe Arg Val Tyr Gly Leu Glu Ser Leu Lys Asp Leu Phe Pro Asn
91 95 100 105
Leu Thr Val Ile Arg Gly Ser Arg Leu Phe Phe Asn Tyr Ala Leu
106 110 115 120
Val Ile Phe Glu Met Val His Leu Lys Glu Leu Gly Leu Tyr Asn
121 125 130 135
Leu Met Asn Ile Thr Arg Gly Ser Val Arg Ile Glu Lys Asn Asn
136 140 145 150
Glu Leu Cys Tyr Leu Ala Thr Ile Asp Trp Ser Arg Ile Leu Asp
151 155 160 165
Ser Val Glu Asp Asn Tyr Ile Val Leu Asn Lys Asp Asp Asn Glu
166 170 175 180
Glu Cys Gly Asp Ile Cys Pro Gly Thr Ala Lys Gly Lys Thr Asn
181 185 190 195
Cys Pro Ala Thr Val Ile Asn Gly Gln Phe Val Glu Arg Cys Trp
196 200 205 210
Thr His Ser His Cys Gln Lys Val Cys Pro Thr Ile Cys Lys Ser
211 215 220 225
His Gly Cys Thr Ala Glu Gly Leu Cys Cys His Ser Glu Cys Leu
226 230 235 240
Gly Asn Cys Ser Gln Pro Asp Asp Pro Thr Lys Cys Val Ala Cys
241 245 250 255
Arg Asn Phe Tyr Leu Asp Gly Arg Cys Val Glu Thr Cys Pro Pro
256 260 265 270
Pro Tyr Tyr His Phe Gln Asp Trp Arg Cys Val Asn Phe Ser Phe
271 275 280 285
Cys Gln Asp Leu His His Lys Cys Lys Asn Ser Arg Arg Gln Gly
286 290 295 300
Cys His Gln Tyr Val Ile His Asn Asn Lys Cys Ile Pro Glu Cys
301 305 310 315
Pro Ser Gly Tyr Thr Met Asn Ser Ser Asn Leu Leu Cys Thr Pro
316 320 325 330
Cys Leu Gly Pro Cys Pro Lys Val Cys His Leu Leu Glu Gly Glu
331 335 340 345
Lys Thr Ile Asp Ser Val Thr Ser Ala Gln Glu Leu Arg Gly Cys
346 350 355 360
Thr Val Ile Asn Gly Ser Leu Ile Ile Asn Ile Arg Gly Gly Asn
361 365 370 375
Asn Leu Ala Ala Glu Leu Glu Ala Asn Leu Gly Leu Ile Glu Glu
376 380 385 390
Ile Ser Gly Tyr Leu Lys Ile Arg Arg Ser Tyr Ala Leu Val Ser
391 395 400 405
Leu Ser Phe Phe Arg Lys Leu Arg Leu Ile Arg Gly Glu Thr Leu
406 410 415 420
Glu Ile Gly Asn Tyr Ser Phe Tyr Ala Leu Asp Asn Gln Asn Leu
421 425 430 435
Arg Gln Leu Trp Asp Trp Ser Lys His Asn Leu Thr Ile Thr Gln
436 440 445 450
Gly Lys Leu Phe Phe His Tyr Asn Pro Lys Leu Cys Leu Ser Glu
451 455 460 465
Ile His Lys Met Glu Glu Val Ser Gly Thr Lys Gly Arg Gln Glu
466 470 475 480
Arg Asn Asp Ile Ala Leu Lys Thr Asn Gly Asp Gln Ala Ser Cys
481 485 490 495
Glu Asn Glu Leu Leu Lys Phe Ser Tyr Ile Arg Thr Ser Phe Asp
496 500 505 510
Lys Ile Leu Leu Arg Trp Glu Pro Tyr Trp Pro Pro Asp Phe Arg
511 515 510 525
Asp Leu Leu Gly Phe Met Leu Phe Tyr Lys Glu Ala Pro Tyr Gln
526 530 535 540
Asn Val Thr Glu Phe Asp Gly Gln Asp Ala Cys Gly Ser Asn Ser
541 545 550 555
Trp Thr Val Val Asp Ile Asp Pro Pro Leu Arg Ser Asn Asp Pro
556 560 565 570
Lys Ser Gln Asn His Pro Gly Trp Leu Met Arg Gly Leu Lys Pro
571 575 580 585
Trp Thr Gln Tyr Ala Ile Phe Val Lys Thr Leu Val Thr Phe Ser
586 590 595 600
Asp Glu Arg Arg Thr Tyr Gly Ala Lys Ser Asp Ile Ile Tyr Val
601 605 610 615
Gln Thr Asp Ala Thr Asn Pro Ser Val Pro Leu Asp Pro Ile Ser
616 620 625 630
Val Ser Asn Ser Ser Ser Gln Ile Ile Leu Lys Trp Lys Pro Pro
631 635 640 645
Ser Asp Pro Asn Gly Asn Ile Thr His Tyr Leu Val Phe Trp Glu
646 650 655 660
Arg Gln Ala Glu Asp Ser Glu Leu Phe Glu Leu Asp Tyr Cys Leu
661 665 670 675
Lys Gly Leu Lys Leu Pro Ser Arg Thr Trp Ser Pro Pro Phe Glu
676 680 685 690
Ser Glu Asp Ser Gln Lys His Asn Gln Ser Glu Tyr Glu Asp Ser
691 695 700 705
Ala Gly Glu Cys Cys Ser Cys Pro Lys Thr Asp Ser Gln Ile Leu
706 710 715 720
Lys Glu Leu Glu Glu Ser Ser Phe Arg Lys Thr Phe Glu Asp Tyr
721 725 730 735
Leu His Asn Val Val Phe Val Pro Arg Lys Thr Ser Ser Gly Thr
736 740 745 750
Gly Ala Glu Asp Pro Arg Pro Ser Arg Lys Arg Arg Ser Leu Gly
751 755 760 765
Asp Val Gly Asn Val Thr Val Ala Val Pro Thr Val Ala Ala Phe
766 770 775 780
Pro Asn Thr Ser Ser Thr Ser Val Pro Thr Ser Pro Glu Glu His
781 785 790 795
Arg Pro Phe Glu Lys Val Val Asn Lys Glu Ser Leu Val Ile Ser
796 800 805 810
Gly Leu Arg His Phe Thr Gly Tyr Arg Ile Glu Leu Gln Ala Cys
811 815 820 825
Asn Gln Asp Thr Pro Glu Glu Arg Cys Ser Val Ala Ala Tyr Val
826 830 835 840
Ser Ala Arg Thr Met Pro Glu Ala Lys Ala Asp Asp Ile Val Gly
841 845 850 855
Pro Val Thr His Glu Ile Phe Glu Asn Asn Val Val His Leu Met
856 860 865 870
Trp Gln Glu Pro Lys Glu Pro Asn Gly Leu Ile Val Leu Tyr Glu
871 875 880 885
Val Ser Tyr Arg Arg Tyr Gly Asp Glu Glu Leu His Leu Cys Val
886 890 895 900
Ser Arg Lys His Phe Ala Leu Glu Arg Gly Cys Arg Leu Arg Gly
901 905 910 915
Leu Ser Pro Gly Asn Tyr Ser Val Arg Ile Arg Ala Thr Ser Leu
916 920 925 930
Ala Gly Asn Gly Ser Trp Thr Glu Pro Thr Tyr Phe Tyr Val Thr
931 935 940 945
Asp Tyr Leu Asp Val Pro Ser Asn Ile Ala Lys Ile Ile Ile Gly
946 950 955 960
Pro Leu Ile Phe Val Phe Leu Phe Ser Val Val Ile Gly Ser Ile
961 965 970 975
Tyr Leu Phe Leu Arg Lys Arg Gln Pro Asp Gly Pro Leu Gly Pro
976 980 985 990
Leu Tyr Ala Ser Ser Asn Pro Glu Tyr Leu Ser Ala Ser Asp Val
991 995 1000 1005
Phe Pro Cys Ser Val Tyr Val Pro Asp Glu Trp Glu Val Ser Arg
1006 1010 1015 1020
Glu Lys Ile Thr Leu Leu Arg Glu Leu Gly Gln Gly Ser Phe Gly
1021 1025 1030 1035
Met Val Tyr Glu Gly Asn Ala Arg Asp Ile Ile Lys Gly Glu Ala
1036 1140 1145 1050
Glu Thr Arg Val Ala Val Lys Thr Val Asn Glu Ser Ala Ser Leu
1051 1155 1160 1065
Arg Glu Arg Ile Glu Phe Leu Asn Glu Ala Ser Val Met Lys Gly
1066 1170 1175 1080
Phe Thr Cys His His Val Val Arg Leu Leu Gly Val Val Ser Lys
1081 1185 1190 1095
Gly Gln Pro Thr Leu Val Val Met Glu Leu Met Ala His Gly Asp
1096 1100 1105 1110
Leu Lys Ser Tyr Leu Arg Ser Leu Arg Pro Glu Ala Glu Asn Asn
1111 1115 1120 1125
Pro Gly Arg Pro Pro Pro Thr Leu Gln Glu Met Ile Gln Met Ala
1126 1130 1135 1140
Ala Glu Ile Ala Asp Gly Met Ala Tyr Leu Asn Ala Lys Lys Phe
1141 1145 1150 1155
Val His Arg Asp Leu Ala Ala Arg Asn Cys Met Val Ala His Asp
1156 1160 1165 1170
Phe Thr Val Lys Ile Gly Asp Phe Gly Met Thr Arg Asp Ile Tyr
1171 1175 1180 1185
Glu Thr Asp Tyr Tyr Arg Lys Gly Gly Lys Gly Leu Leu Pro Val
1186 1190 1195 1200
Arg Trp Met Ala Pro Glu Ser Leu Lys Asp Gly Val Phe Thr Thr
1201 1205 1210 1215
Ser Ser Asp Met Trp Ser Phe Gly Val Val Leu Trp Glu Ile Thr
1216 1220 1225 1230
Ser Leu Ala Glu Gln Pro Tyr Gln Gly Leu Ser Asn Glu Gln Val
1231 1235 1240 1245
Leu Lys Phe Val Met Asp Gly Gly Tyr Leu Asp Gln Pro Asp Asn
1246 1250 1255 1260
Cys Pro Glu Arg Val Thr Asp Leu Met Arg Met Cys Trp Gln Phe
1261 1265 1270 1275
Asn Pro Lys Met Arg Pro Thr Phe Leu Glu Ile Val Asn Leu Leu
1276 1280 1285 1290
Lys Asp Asp Leu His Pro Ser Phe Pro Glu Val Ser Phe Phe His
1291 1295 1300 1305
Ser Glu Glu Asn Lys Ala Pro Glu Ser Glu Glu Leu Glu Met Glu
1306 1310 1315 1320
Phe Glu Asp Met Glu Asn Val Pro Leu Asp Arg Ser Ser His Cys
1321 1325 1330 1335
Gln Arg Glu Glu Ala Gly Gly Arg Asp Gly Gly Ser Ser Leu Gly
1336 1340 1345 1350
Phe Lys Arg Ser Tyr Glu Glu His Ile Pro Tyr Thr His Met Asn
1351 1355 1360 1365
Gly Gly Lys Lys Asn Gly Arg Ile Leu Thr Leu Pro Arg Ser Asn
1366 1370 1375 1380
Pro Ser
13811382
```

The exemplary procedure for preparation of the starting polyclonal antibodies to C-terminal fragment of beta subunit of human insulin receptor may be described as follows. In 7-9 days before blood sampling, 1-3 intravenous injections of the desired antigen are made to the rabbits to increase the level of polyclonal antibodies in the rabbit blood stream. Upon immunization, blood samples are taken to test the antibody level. Typically, the maximum level of immune reaction of the soluble antigen is achieved within 40 to 60 days after the first injection of the antigen. Upon completion of the first immunization cycle, rabbits have a 30-day rehabilitation period, after which re-immunization is performed with another 1-3 intravenous injections.

To obtain antiserum containing the desired antibodies, the immunized rabbits' blood is collected from rabbits and placed in 50 ml centrifuge tube. Product clots formed on the tube sides are removed with a wooden spatula, and a rod is placed into the clot in the tube center. The blood is then placed in a refrigerator for one night at the temperature of about 40° C. On the following day, the clot on the spatula is removed, and the remaining liquid is centrifuged for 10 min at 13,000 rotations. Supernatant fluid is the target antiserum. The obtained antiserum is typically yellow. 20% of NaN₃ (weight concentration) is added in the antiserum to the final concentration of 0.02% and stored before use in frozen state at the temperature of −20° C. (or without NaN₃ at the temperature of −70° C.). To separate the target antibodies to C-terminal fragment of beta subunit of human insulin-receptor from the antiserum, the following solid phase absorption sequence is suitable:

10 ml of the antiserum of rabbits is diluted twofold with 0.15 M NaCl, after which 6.26 g Na₂SO₄ is added, mixed and incubated for 12-16 hours at 4° C. The sediment is removed by centrifugation, diluted in 10 ml of phosphate buffer and dialyzed against the same buffer during one night at ambient temperature. After the sediment is removed, the solution is applied to DEAE-cellulose column balanced by phosphate buffer. The antibody fraction is determined by measuring the optical density of eluate at 280 Nm.

The isolated crude antibodies are purified using the affine chromatography method by attaching the obtained antibodies to a C-terminal fragment of beta subunit of human insulin receptor located on the insoluble matrix of the chromatography media, with subsequent elution by concentrated aqueous salt solutions.

The resulting buffer solution is used as the initial solution for the homeopathic dilution process used to prepare the activated potentiated form of the antibodies. The preferred concentration of the initial matrix solution of the antigen-purified polyclonal rabbit antibodies to C-terminal fragment of beta subunit of human insulin-receptor is 0.5 to 5.0 mg/ml, preferably, 2.0 to 3.0 mg/ml.

In order to obtain polyclonal antibodies to human interferon gamma, it is possible to use the adjuvant and, for example, the intact molecule of interferon gamma of the below-described sequence as immunogen (antigen) for rabbit immunization:

```
SEQ ID NO: 15
Met Lys Tyr Thr Ser Tyr Ile Leu Ala Phe Gln Leu Cys Ile Val
  1 5 10 15
Leu Gly Ser Leu Gly Cys Tyr Cys Gln Asp Pro Tyr Val Lys Glu
  16 20 25 30
Ala Glu Asn Leu Lys Lys Tyr Phe Asn Ala Gly His Ser Asp Val
  31 35 40 45
Ala Asp Asn Gly Thr Leu Phe Leu Gly Ile Leu Lys Asn Trp Lys
  46 50 55 60
Glu Glu Ser Asp Arg Lys Ile Met Gln Ser Gln Ile Val Ser Phe
  61 65 70 75
Tyr Phe Lys Leu Phe Lys Asn Phe Lys Asp Asp Gln Ser Ile Gln
  76 80 85 90
Lys Ser Val Glu Thr Ile Lys Glu Asp Met Asn Val Lys Phe Phe
  91 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro Ala Ala Lys Thr Gly
136 140 145 150
Lys Arg Lys Arg Ser Gln Met Leu Phe Arg Gly Arg Arg Ala Ser
151 155 160 165
Gln
166
```

In order to obtain polyclonal antibodies to human interferon gamma, it is possible to use the adjuvant and, for example, a polypeptide fragment of human interferon gamma as immunogen (antigen) for rabbit immunization, selected from the following sequences:

```
SEQ ID NO: 16
Ile Leu Ala Phe Gln Leu Cys Ile Val
  7 10 15
Leu Gly Ser Leu Gly Cys Tyr Cys Gln Asp Pro Tyr Val Lys Glu
  16 20 25 30
Ala Glu Asn Leu Lys Lys Tyr Phe Asn Ala Gly His Ser Asp Val
  31 35 40 45
Ala Asp Asn Gly Thr Leu Phe Leu Gly Ile
  46 50 55
SEQ ID NO: 17
Gln Asp Pro Tyr Val Lys Glu
24 30
Ala Glu Asn Leu Lys Lys Tyr Phe Asn Ala Gly His Ser Asp Val
  31 35 40 45
Ala Asp Asn Gly Thr Leu Phe Leu Gly Ile Leu Lys Asn Trp Lys
  46 50 55 60
Glu Glu Ser Asp Arg Lys Ile Met Gln Ser Gln Ile Val Ser Phe
  61 65 70 75
Tyr Phe Lys Leu Phe Lys Asn Phe Lys Asp Asp Gln Ser Ile Gln
  76 80 85 90
Lys Ser Val Glu Thr Ile Lys Glu Asp Met Asn Val Lys Phe Phe
91 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro Ala Ala Lys Thr Gly
136 140 145 150
Lys Arg Lys Arg Ser Gln Met Leu Phe Arg Gly Arg Arg Ala Ser
151 155 160 165
Gln
166
SEQ ID NO: 18
Gln Asp Pro Tyr Val Lys Glu
24 30
Ala Glu Asn Leu Lys Lys Tyr Phe Asn Ala Gly His Ser Asp Val
  31 35 40 45
Ala Asp Asn Gly Thr Leu Phe Leu Gly Ile Leu Lys Asn Trp Lys
  46 50 55 60
Glu Glu Ser Asp Arg Lys Ile Met Gln Ser Gln Ile Val Ser Phe
  61 65 70 75
Tyr Phe Lys Leu Phe Lys Asn Phe Lys Asp Asp Gln Ser Ile Gln
76 80 85 90
Lys Ser Val Glu Thr Ile Lys Glu Asp Met Asn Val Lys Phe Phe
91 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro Ala Ala Lys Thr Gly
136 140 145 150
Lys Arg Lys Arg Ser Gln Met Leu Phe Gln Gly Arg Arg Ala Ser
151 155 160 165
Gln
166
SEQ ID NO: 19
Gln Ser Gln Ile Val Ser Phe
  69 75
Tyr Phe Lys Leu Phe lys Asn Phe Lys Asp Asp Gln Ser Ile Gln
76 80 85 90
Lys Ser Val Glu Thr Ile Lys Glu Asp Met Asn Val Lys Phe Phe
  91 95 100 105
Asn Ser Asn Lys Lys Lys Arg Asp Asp Phe Glu Lys Leu Thr Asn
106 110 115 120
Tyr Ser Val
121 123
SEQ ID NO: 20
Met Asn Val Lys Phe Phe
100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro
136 140 145
SEQ ID NO: 21
Ser Val Glu Thr Ile Lys Glu Asp Met Asn Val Lys Phe Phe
92 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg
121 125 130
SEQ ID NO: 22
Val Thr Asp Leu Asn Val Gln Arg Lys Ala Ile His Glu
  123 125 130 135
Leu Ile Gln Val Met Ala Glu Leu Ser Pro Ala Ala
136 140 145 147
SEQ ID NO: 23
Ser Tyr Ile Leu Ala Phe Gln Leu Cys Ile Val
    5 10 15
Leu Gly Ser Leu Gly Cys Tyr Cys Gln Asp Pro Tyr Val Lys Glu
  16 20 25 30
Ala Glu Asn Leu Lys Lys Tyr Phe Asn Ala Gly His Ser Asp Val
  31 35 40 45
SEQ ID NO: 24
Glu Thr Ile Lys Glu Asp Met Asn Val Lys Phe Phe
  94 100 105
Asn Ser Asn Lys Lys Lys Arg Asp Asp
106 110 114
```

The polyclonal antibodies to human interferon gamma may be obtained using molecules of recombinant interferon gamma of the below-described sequence:

```
SEQ ID NO: 25
Met Gln Asp Pro Tyr Val Lys Glu
24 30
Ala Glu Asn Leu Lys Lys Tyr Phe Asn Ala Gly His Ser Asp Val
  31 35 40 45
Ala Asp Asn Gly Thr Leu Phe Leu Gly Ile Leu Lys Asn Trp Lys
  46 50 55 60
Glu Glu Ser Asp Arg Lys Ile Met Gln Ser Gln Ile Val Ser Phe
61 65 70 75
Tyr Phe Lys Leu Phe Lys Asn Phe Lys Asp Asp Gln Ser Ile Gln
  76 80 85 90
Lys Ser Val Glu Thr Ile Lys Glu Asp Met Asn Val Lys Phe Phe
  91 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro Ala Ala Lys Thr Gly
136 140 145 150
Lys Arg Lys Arg Ser Gln Met Leu Phe Gln Gly Arg Arg Ala Ser
151 155 160 165
Gln
166
SEQ ID NO: 26
24 30
Ala Glu Asn Leu Lys Lys Tyr Phe Asn Ala Gly His Ser Asp Val
  31 35 40 45
Ala Asp Asn Gly Thr Leu Phe Leu Gly Ile Leu Lys Asn Trp Lys
  46 50 55 60
Glu Glu Ser Asp Arg Lys Ile Met Gln Ser Gln Ile Val Ser Phe
61 65 70 75
Tyr Phe Lys Leu Phe Lys Asn Phe Lys Asp Asp Gln Ser Ile Gln
  76 80 85 90
Lys Ser Val Glu Thr Ile Lys Glu Asp Met Asn Val Lys Phe Phe
  91 95 100 105
Asn Ser Asn Lys Lys Lys Arg Asp Asp Phe Glu Lys Leu Thr Asn
106 110 115 120
Tyr Ser Val Thr Asp Leu Asn Val Gln Arg Lys Ala Ile His Glu
121 125 130 135
Leu Ile Gln Val Met Ala Glu Leu Ser Pro Ala Ala Lys Thr Gly
136 140 145 150
Lys Arg Lys Arg Ser Gln Met Leu Phe Arg Gly Arg Arg Ala Ser
151 155 160 165
Gln
166
```

The polyclonal antibodies to CD4 receptor are obtained by a similar methodology using the adjuvant and, for example, the entire molecule or a polypeptide fragment of CD4 receptor as immunogen (antigen) for rabbit immunization, selected, for example, from the following sequences:

```
SEQ. ID. NO. 27
Met Asn Arg Gly Val Pro Phe Arg His Leu Leu Leu Val Leu Gln
  1 5 10 15
Leu Ala Leu Leu Pro Ala Ala Thr Gln Gly Lys Lys Val Val Leu
  16 20 25 30
Gly Lys Lys Gly Asp Thr Val Glu Leu Thr Cys Thr Ala Ser Gln
  31 35 40 45
Lys Lys Ser Ile Gln Phe His Trp Lys Asn Ser Asn Gln Ile Lys
  46 50 55 60
Ile Leu Gly Asn Gln Gly Ser Phe Leu Thr Lys Gly Pro Ser Lys
  61 65 70 75
Leu Asn Asp Arg Ala Asp Ser Arg Arg Ser Leu Trp Asp Gln Gly
  76 80 85 90
Asn Phe Pro Leu Ile Ile Lys Asn Leu Lys Ile Glu Asp Ser Asp
  91 95 100 105
Thr Tyr Ile Cys Glu Val Glu Asp Gln Lys Glu Glu Val Gln Leu
106 110 115 120
Leu Val Phe Gly Leu Thr Ala Asn Ser Asp Thr His Leu Leu Gln
121 125 130 135
Gly Gln Ser Leu Thr Leu Thr Leu Glu Ser Pro Pro Gly Ser Ser
136 140 145 150
Pro Ser Val Gln Cys Arg Ser Pro Arg Gly Lys Asn Ile Gln Gly
151 155 160 165
Gly Lys Thr Leu Ser Val Ser Gln Leu Glu Leu Gln Asp Ser Gly
166 170 175 180
Thr Trp Thr Cys Thr Val Leu Gln Asn Gln Lys Lys Val Glu Phe
181 185 190 195
Lys Ile Asp Ile Val Val Leu Ala Phe Gln Lys Ala Ser Ser Ile
196 200 205 210
Val Tyr Lys Lys Glu Gly Glu Gln Val Glu Phe Ser Phe Pro Leu
211 215 220 225
Ala Phe Thr Val Glu Lys Leu Thr Gly Ser Gly Glu Leu Trp Trp
226 230 235 240
Gln Ala Glu Arg Ala Ser Ser Ser Lys Ser Trp Ile Thr Phe Asp
241 245 250 255
Leu Lys Asn Lys Glu Val Ser Val Lys Arg Val Thr Gln Asp Pro
256 260 265 270
Lys Leu Gln Met Gly Lys Lys Leu Pro Leu His Leu Thr Leu Pro
271 275 280 285
Gln Ala Leu Pro Gln Tyr Ala Gly Ser Gly Asn Leu Thr Leu Ala
286 290 295 300
Leu Glu Ala Lys Thr Gly Lys Leu His Gln Glu Val Asn Leu Val
301 305 310 315
Val Met Arg Ala Thr Gln Leu Gln Lys Asn Leu Thr Cys Glu Val
316 320 325 330
Trp Gly Pro Thr Ser Pro Lys Leu Met Leu Ser Leu Lys Leu Glu
331 335 340 345
Asn Lys Glu Ala Lys Val Ser Lys Arg Glu Lys Ala Val Trp Val
346 350 355 360
Leu Asn Pro Glu Ala Gly Met Trp Gln Cys Leu Leu Ser Asp Ser
361 365 370 375
Gly Gln Val Leu Leu Glu Ser Asn Ile Lys Val Leu Pro Thr Trp
376 380 385 390
Ser Thr Pro Val Gln Pro Met Ala Leu Ile Val Leu Gly Gly Val
391 395 400 405
Ala Gly Leu Leu Leu Phe Ile Gly Leu Gly Ile Phe Phe Cys Val
406 410 415 420
Arg Cys Arg His Arg Arg Arg Gln Ala Glu Arg Met Ser Gln Ile
421 425 430 435
Lys Arg Leu Leu Ser Glu Lys Lys Thr Cys Gln Cys Pro His Arg
436 440 445 450
Phe Gln Lys Thr Cys Ser Pro Ile
451 445 458
SEQ. ID. NO. 28
Ile Gly Leu Gly Ile Phe Phe Cys Val
412 415 420
Arg Cys Arg His Arg Arg Arg Gln Ala Glu Arg Met Ser Gln Ile
421 425 430 435
Lys Arg Leu Leu Ser Glu Lys Lys Thr Cys Gln Cys Pro His Arg
436 440 445 450
Phe Gln Lys Thr Cys Ser Pro Ile
451 445 458
SEQ. ID. NO. 29
Gly Lys Lys Val Val Leu
  26 30
Gly Lys Lys Gly Asp Thr Val Glu Leu Thr Cys Thr Ala Ser Gln
  31 35 40 45
Lys Lys Ser Ile Gln Phe His Trp Lys Asn Ser Asn Gln Ile Lys
  46 50 55 60
SEQ. ID. NO. 30
                                                                                                                Asp
  91 95 100 105
Thr Tyr Ile Cys Glu Val Glu Asp Gln Lys Glu Glu Val Gln
106 110 115 119
SEQ ID NO: 31
Lys Glu Glu Val Gln Leu
115 120
Leu Val Phe Gly Leu Thr Ala Asn Ser Asp Thr His Leu Leu Gln
121 125 130 135
Gly Gln Ser Leu
136 139
SEQ ID NO: 32
Gly Lys Lys Val Val Leu
  26 30
Gly Lys Lys Gly Asp Thr Val Glu Leu Thr Cys Thr Ala Ser Gln
  31 35 40 45
Lys Lys Ser Ile Gln Phe His Trp Lys Asn Ser Asn Gln Ile Lys
  46 50 55 60
Ile Leu Gly Asn Gln Gly Ser Phe Leu Thr Lys Gly Pro Ser Lys
  61 65 70 75
Leu Asn Asp Arg Ala Asp Ser Arg Arg Ser Leu Trp Asp Gln Gly
  76 80 85 90
Asn Phe Pro Leu Ile Ile Lys Asn Leu Lys Ile Glu Asp Ser Asp
  91 95 100 105
Thr Tyr Ile Cys Glu Val Glu Asp Gln Lys Glu Glu Val Gln Leu
106 110 115 120
Leu Val Phe Gly Leu Thr Ala Asn Ser Asp Thr His Leu Leu Gln
121 125 130 135
Gly Gln Ser Leu Thr Leu Thr Leu Glu Ser Pro Pro Gly Ser Ser
136 140 145 150
Pro Ser Val Gln Cys Arg Ser Pro Arg Gly Lys Asn Ile Gln Gly
151 155 160 165
Gly Lys Thr Leu Ser Val Ser Gln Leu Glu Leu Gln Asp Ser Gly
166 170 175 180
Thr Trp Thr Cys Thr Val Leu Gln Asn Gln Lys Lys Val Glu Phe
181 185 190 195
Lys Ile Asp Ile Val Val Leu Ala Phe Gln Lys Ala Ser Ser Ile
196 200 205 210
Val Tyr Lys Lys Glu Gly Glu Gln Val Glu Phe Ser Phe Pro Leu
211 215 220 225
Ala Phe Thr Val Glu Lys Leu Thr Gly Ser Gly Glu Leu Trp Trp
226 230 235 240
Gln Ala Glu Arg Ala Ser Ser Ser Lys Ser Trp Ile Thr Phe Asp
241 245 250 255
Leu Lys Asn Lys Glu Val Ser Val Lys Arg Val Thr Gln Asp Pro
256 260 265 270
Lys Leu Gln Met Gly Lys Lys Leu Pro Leu His Leu Thr Leu Pro
271 275 280 285
Gln Ala Leu Pro Gln Tyr Ala Gly Ser Gly Asn Leu Thr Leu Ala
286 290 295 300
Leu Glu Ala Lys Thr Gly Lys Leu His Gln Glu Val Asn Leu Val
301 305 310 315
Val Met Arg Ala Thr Gln Leu Gln Lys Asn Leu Thr Cys Glu Val
316 320 325 330
Trp Gly Pro Thr Ser Pro Lys Leu Met Leu Ser Leu Lys Leu Glu
331 335 340 345
Asn Lys Glu Ala Lys Val Ser Lys Arg Glu Lys Ala Val Trp Val
346 350 355 360
Leu Asn Pro Glu Ala Gly Met Trp Gln Cys Leu Leu Ser Asp Ser
361 365 370 375
Gly Gln Val Leu Leu Glu Ser Asn Ile Lys Val Leu Pro Thr Trp
376 380 385 390
Ser Thr Pro Val Gln Pro Met Ala Leu Ile Val Leu Gly Gly Val
391 395 400 405
Ala Gly Leu Leu Leu Phe Ile Gly Leu Gly Ile Phe Phe Cys Val
406 410 415 420
Arg Cys Arg His Arg Arg Arg ln Ala lu Arg Met er Gln Ile
421 425 430 435
Lys Arg Leu Leu Ser Glu Lys Lys Thr Cys Gln Cys Pro His Arg
436 440 445 450
Phe Gln Lys Thr Cys Ser Pro Ile
451 445 458
```

The activated potentiated form of each component of the veterenary composition may be prepared from initial solution by homeopathic potentization, preferably using the method of proportional concentration decrease by serial dilution of 1 part of each preceding solution (beginning with the initial solution) in 9 parts (for decimal dilution), or in 99 parts (for centesimal dilution), or in 999 parts (for millesimal dilution) of a neutral solvent, coupled with external impact. Preferably, the external impact involves multiple vertical shaking (dynamization) of each dilution. Preferably, separate containers are used for each subsequent dilution up to the required potency level, or the dilution factor. This method is well-accepted in the homeopathic art. See, e.g. V. Schwabe “Homeopathic medicines”, M., 1967, p. 14-29, incorporated herein by reference for the purpose stated.

For example, to prepare a 12-centesimal dilution (denoted C12), one part of the initial matrix solution of antibodies to C-terminal fragment of beta subunit of human insulin receptor with the concentration of 3.0 mg/ml is diluted in 99 parts of neutral aqueous or aqueous-alcohol solvent (preferably, 15%-ethyl alcohol) and then vertically shaken many times (10 and more) to create the 1st centesimal dilution (denoted as C1). The 2nd centesimal dilution (C2) is prepared from the 1st centesimal dilution C1. This procedure is repeated 11 times to prepare the 12th centesimal dilution C12. Thus, the 12th centesimal dilution C12 represents a solution obtained by 12 serial dilutions of one part of the initial matrix solution of antibodies to C-terminal fragment of beta subunit of human insulin-receptor with the concentration of 3.0 mg/ml in 99 parts of a neutral solvent in different containers, which is equivalent to the centesimal homeopathic dilution C12. Similar procedures with the relevant dilution factor are performed to obtain dilutions C30 and C 200. The intermediate dilutions may be tested in a desired biological model to check activity. The preferred activated potentiated forms for both antibodies comprising the combination of the invention are a mixture of C12, C30, and C200 dilutions. When using the mixture of various homeopathic dilutions (primarily centesimal) of the active substance as biologically active liquid component, each component of the composition (e.g., C12, C30, C200) is prepared separately according to the above-described procedure until the next-to-last dilution is obtained (e.g., until C11, C29, and C199 respectively), and then one part of each component is added in one container according to the mixture composition and mixed with the required quantity of the solvent (e.g. with 97 parts for centesimal dilution).

It is possible to use the active substance as mixture of various homeopathic dilutions, e.g. decimal and/or centesimal (D 20, C 30, C100 or C12, C30, C50 etc.), the efficiency of which is determined experimentally by testing the dilution in a suitable biological model, for example, in models described in the examples herein.

In course of potentiation and concentration decrease, the vertical shaking may be substituted for external exposure to ultrasound, electromagnetic field or any similar external impact procedure accepted in the homeopathic art.

Preferably, the pharmaceutical composition of the invention may be in the form of a liquid or in the solid unit dosage form. The preferred liquid form of the pharmaceutical composition is a mixture, preferably, at a 1:1 ratio of the activated potentiated form of antibodies. The preferred liquid carrier is water or water-ethyl alcohol mixture.

The solid unit dosage form of the pharmaceutical composition of the invention may be prepared by impregnating a solid, pharmaceutically acceptable carrier with the mixture of the activated potentiated form of aqueous or aqueous-alcohol solutions of active components. Alternatively, the carrier may be impregnated consecutively with each requisite dilution. Both orders of impregnation are acceptable.

Preferably, to prepare the claimed veterinary composition in a form of a compound drug, the aqueous or aqueous-alcoholic solutions of the active components are mixed (primarily in 1:1:1 ratio by volume) and used in a liquid dosage form.

The veterinary composition of the invention may also be in a solid unit dosage form (formulated as a powder or tablet) and represent a compound drug containing a technologically required (efficient) amount of a neutral carrier (e.g. lactose) saturated by impregnation with, for example, a mixture of aqueous or aqueous-alcohol solutions of the activated-potentiated form of antibodies to the insulin receptor β-subunit (antibodies to a C-terminal fragment of the insulin receptor β-subunit), the activated-potentiated form of antibodies to human interferon gamma, and activated-potentiated form of antibodies to CD4 receptor in combination with pharmaceutically acceptable excipients, primarily including lactose, microcrystalline cellulose and magnesium stearate.

Preferably, the pharmaceutical composition in the solid unit dosage form is prepared from granules of the pharmaceutically acceptable carrier which was previously saturated with the aqueous or aqueous-alcoholic dilutions of the activated potentiated form of antibodies to C-terminal fragment of beta subunit of human insulin-receptor. The solid dosage form may be in any form known in the pharmaceutical art, including a tablet, a capsule, a lozenge, and others. As an inactive pharmaceutical ingredients one can use glucose, sucrose, maltose, amylum, isomaltose, isomalt and other mono- olygo- and polysaccharides used in manufacturing of pharmaceuticals as well as technological mixtures of the above mentioned inactive pharmaceutical ingredients with other pharmaceutically acceptable excipients, for example isomalt, crospovidone, sodium cyclamate, sodium saccharine, anhydrous citric acid etc), including lubricants, disintegrants, binders and coloring agents. The preferred carriers are lactose and isomalt. The pharmaceutical dosage form may further include standard pharmaceutical excipients, for example, microcrystalline cellulose and magnesium stearate.

To prepare the solid oral form formulated as a tablet, 50-500 μm granules of the neutral excipient—lactose (milk sugar), which were previously saturated with an aqueous or aqueous-alcoholic solution of the activated-potentiated form of antibodies to the insulin receptor β-subunit (or, for example, antibodies to insulin receptor β-subunit, to human interferon gamma, and to CD4 receptor) in the ratio of 1 kg of antibody solution to 5 or 10 kg of lactose (1:5 to 1:10), are exposed to saturation irrigation in the fluidized boiling bed in a fluid bed system (e.g. “Hüttlin Pilotlab” by Hüttlin GmbH) with subsequent drying with preheated air flow introduced through the bed plate at a temperature below 40° C. The estimated amount of the lactose (10÷ 91% of the tablet mass (by weight)) saturated with the activated-potentiated form of antibodies according to the above-described processing procedure is loaded in the mixer hopper, and mixed with lactose saturated with the activated-potentiated form of antibodies taken at the amount of 3 to 10 weight parts (3÷10% of the tablet mass) and with no more than 84 weight parts (81% of the tablet mass) of “non-saturated” pure lactose (used for the purposes of cost reduction and simplification and acceleration of the technological process without decreasing the treatment efficiency). Then the mixture is supplemented with 5 to 10 weight parts (5÷10% of the tablet mass) of cellulose and 1 weight part 1% of the tablet mass) of magnesium stearate. The obtained tablet mass is uniformly mixed, and tableted by direct dry pressing (e.g., in a Korsch—XL 400 tablet press) to form 150 to 500 mg round pills. After tableting, 300 mg pills are obtained that are saturated with aqueous-alcoholic solution (3.0-6.0 mg/pill) of the activated-potentiated form of antibodies to the insulin receptor β-subunit (or, for example, antibodies to insulin receptor β-subunit, to human interferon gamma, and to CD4 receptor). Each component of the combination used to impregnate the carrier is in an ultra-low dose prepared from the initial matrix solution diluted by a factor of 100¹², 100³⁰ and 100⁵⁰, which is equivalent to a mixture of centesimal homeopathic dilutions C12, C30 and C50.

While the invention is not limited to any specific theory, it is believed that the activated potentiated form of the antibodies described herein do not contain the molecular form of the antibody in the amount sufficient to have biological activity attributed to such molecular form. The biological activity of the composition of the invention is amply demonstrated in the appended examples.

The composition of the invention may be used for improving livability of animals, primarily, promoting live-weight gain and growth of mammals and birds (preferably food-producing animals and poultry), enhancing the effectiveness of immunization, preventing and/or treating a broad range of diseases (including infectious diseases of various etiology), and increasing livestock performance, reproduction and survival.

### EXAMPLES

### Example 1

The effect of the claimed compound intended for promoting body weight gain in mammals and birds, enhancing the effectiveness of immunization, and preventing and/or treating infectious diseases, in the form of aqueous solution containing an activated-potentiated form of antigen-purified ultra-low dose polyclonal rabbit antibodies to the insulin receptor β-subunit (prepared by extreme dilution of the primary matrix solution (concentration of 2.5 mg/ml) by a factor of 100¹², 100³⁰, 100²⁰⁰), which is equivalent to a mixture of centesimal homeopathic C12, C30 and C200 dilutions (anti-IRβ Ab), on body weight changes was evaluated in mature male albino Wistar rats ( ). The test compound was administered intragastrically (via a gavage needle) at 2.5 ml/kg once daily for 6 months (n=20). The control animals were dosed in a similar manner with 2.5 ml/kg of settled water (n=20). The overall study duration, including a period of one month after treatment discontinuation, was 7 months. General health and body weight changes of the animals were recorded regularly at monthly intervals.

There were no differences in general health assessments between the animal groups throughout the study period: the animals did not show restlessness or changes in appetite, defecation, and state of the mucosa, hair and skin, etc. Body weight data at different monitoring time points are summarized in Table 1. There was an increase (p>0.05) observed as soon as at the end of the second month of monitoring in the weight gain values of animals receiving RA anti-IRβ Ab compared to the control group. At 3, 4, 5 and 6 months of the dosing period, the rats' body weights were significantly incremented in the RA anti-IRβ group as compared to control animals. The noted body weight increases as related to the control group were 6.1%, 9.4%, 10.4% and 11.2% at 3, 4, 5 and 6 months of the dosing period, respectively. Following one month after treatment discontinuation, the rats' body weights in the RA anti-IRβ group remained increased as compared to control values (p>0.05).

TABLE 1
Body weight changes of male Wistar rats

| | Control | anti-IRβ Ab |
|---|---|---|
| Month 1 | 216.75 ± 3.96 | 204.75 ± 5.05 |
| Month 2 | 233.75 ± 4.23 | 242.00 ± 3.72 |
| Month 3 | 256.50 ± 4.11 | 272.25 ± 4.3* |
| Month 4 | 269.00 ± 4.24 | 294.21 ± 3.98* |
| Month 5 | 280.00 ± 3.72 | 309.21 ± 4.64* |
| Month 6 | 289.25 ± 3.43 | 321.58 ± 4.81* |
| Month 1 post- | 318.33 ± 6.94 | 331.82 ± 6.75 |
| discontinuation | | |

*p > 0.05 compared to controls

### Example 2

The effect of the claimed compound (Preparation 1) for promoting body weight gain in mammals and birds, enhancing the effectiveness of immunization, and preventing and/or treating infectious diseases, in the form of aqueous solution containing an activated-potentiated form of antigen-purified ultra-low dose polyclonal rabbit antibodies to a C-terminal fragment of the insulin receptor β-subunit (prepared by extreme dilution of the primary matrix solution (concentration of 2.5 mg/ml) by a factor of 100¹², 100³⁰, 100²⁰⁰), which is equivalent to a mixture of centesimal homeopathic C12, C30 and C200 dilutions (ant-IRβ Ab), on performance and body weight gain was evaluated in Cobb 500 broiler chickens reared in R-15 battery brooders (35 co-housed male and female chickens, aged between 1 to 37 days, in each cage) at the brooder house of the Zagorsk Experimental Livestock Farm, National Poultry Research and Development Institute (VNITIP), Russian Academy of Agricultural Sciences. Sex ratios were defined in all broiler groups at the end of the breeding period—at 35 or 37 days of age. The testing was conducted in accordance with Guidelines for Broiler Meat Production (Sergiev Posad, 2008). Data on study design and treatment groups are provided in Table 2.

TABLE 2

| | Number | | Daily | Route of | |
|---|---|---|---|---|---|
| | of | | dosage, | adminis- | |
| Group | chicks | Treatment | ml/chick | tration | Regimen |
| 1 | 35 | Prepara- | 20 | with | twice daily |
| | | tion 1 | | drinking | (10 ml/ |
| | | | | water | chick × 2), |
| | | | | | from day 7 |
| | | | | | through day 37 |
| | | | | | of age |
| | | | | | (25 days) |
| 2 | 35 | Prepara- | 20 | with | twice daily |
| | | tion 2 | | drinking | (10 ml/ |
| | | | | water | chick × 2), |
| | | | | | from day 7 |
| | | | | | through day 37 |
| | | | | | of age |
| | | | | | (25 days) |

Within the study, the effect of the claimed compound for weight gain promotion (Preparation 1) was compared with a placebo (distilled water, Preparation 2).

The following study parameters were examined:

1. Body weights of all reared birds as examined on days 1, 7, 14, 28, 35, and 37 of life;

2. Average daily gain in broilers over 14- and 28-day breeding periods (aggregate female and male data), and for 35- and 37-day periods (in aggregate and for separate sexes);

3. Survival rate (%) in the broiler flock—by recording the daily number of deaths and specifying causality;

4. Daily food consumption in the groups and feed conversion (feed intake per 1 kg weight gain) as calculated on days 35 and 37 of the breeding period;

5. Blood samples were collected at slaughter from 37-day-old broilers. The samples were derived from 15 chickens in each group (both treatment and control) and delivered for further analysis to the Russian National Centre for Standardization and Testing of Veterinary Drugs and Feeds (VGNKI) (Moscow, Russia).

6. Based on the growth performance figures, European broiler Index was calculated for each group.

Vaccination of broiler chickens was performed using the following shedule:
at hatchery (day-old chicks)—against Newcastle disease virus, avian infectious bronchitis (aerosol inhalation), and Marek's disease (neurolymphomatosis) (intramuscularly);
at 13 days of age—against infectious bursal disease (IBD) (with drinking water);
at 19 days of age—repeated vaccine against Newcastle disease virus and infectious bronchitis (aerosol inhalation);

To perform data analysis, methods of variance statistics were utilized. For each sampled group P, the mean (M), standard error of the mean (m), standard deviation (a) and coefficient of variation (Cv) were calculated. Normalized skewness and kurtosis values were used to test the normal distribution. In the case of distribution normality, the Student's t-test was employed to compare the sample means (Plokhinsky N. A., 1978).

The productivity results obtained in the study groups of Cobb 500 broilers are presented in Tables 3-6.

Broiler body weight data (g) are gathered in Table 10.

As seen from Table 3, the body weights of 7-day-old chicks were similar in the two study groups.

The body weight values of 14-day-old broilers were 1.16% higher in group 1 then in group 2.

In 28-day-old chicks, the body weights were increased by 3.55% in group 1 as compared to group 2.

TABLE 3

| | | | | | |
|---|---|---|---|---|---|
| Group | n | M | m | σ | Cv |

one day chicks

| | | | | | |
|---|---|---|---|---|---|
| 1 | 35 | 47.69 | 0.67 | 3.96 | 8.3 |
| 2 | 35 | 47.17 | 0.54 | 3.17 | 6.7 |

7-day-old chicks

| | | | | | |
|---|---|---|---|---|---|
| 1 | 35 | 138.09 | 2.39 | 14.15 | 10.3 |
| 2 | 35 | 137.54 | 1.52 | 9.00 | 6.5 |

14-day-old chicks

| | | | | | |
|---|---|---|---|---|---|
| 1 | 35 | 360.71 | 7.59 | 44.87 | 12.4 |
| 2 | 35 | 356.57 | 9.40 | 55.58 | 15.6 |

28-day-old chicks

| | | | | | |
|---|---|---|---|---|---|
| 1 | 35 | 1267.60 | 17.54 | 103.77 | 8.2 |
| 2 | 35 | 1224.20 | 10.67 | 116.39 | 9.5 |

In 35-day-old chicks (Table 4), the body weights in group 1 were 3.22% higher than in the comparator group 2.

Prior to data analysis, the 35-day-old chicks in all groups were sorted by sex. After such division, it was seen that the body weight changes in group1 were mostly associated with changes in the body weights of pullets (Table 5), which demonstrated statistically significant weight increases (Table 4). This trend was still observed on day 37 (Tables 4 and 5).

TABLE 4
Body weights of broiler chickens (g)

| | | | | | |
|---|---|---|---|---|---|
| Group | n | M | m | δ | Cv |

35-day-old chicks

| 1 | 35 | | 1830.97 | | 26.84 | | 158.76 | | 8.7 | |
|---|---|---|---|---|---|---|---|---|---|---|
| 2 | 35 | | 1773.80 | | 25.49 | | 150.82 | | 8.5 | |
| | | | [symbol] | [symbol] | [symbol] | [symbol] | [symbol] | [symbol] | | |
| 1 | 2 | 3 | 1871.83 | 1809.365* | 40.66 | 34.67 | 140.83 | 166.2 | .5 | .2 |
| 2 | 3 | 2 | 1871.77 | 1715.91 | 31.67 | 30.10 | 114.17 | 141.17 | .1 | .2 |

37-day-old chicks

| 1 | 35 | | 1964.83 | | 30.61 | | 181.09 | | 9.2 | |
|---|---|---|---|---|---|---|---|---|---|---|
| 2 | 35 | | 1927.49 | | 27.43 | | 162.26 | | 8.4 | |
| | | | [symbol] | [symbol] | [symbol] | [symbol] | [symbol] | [symbol] | | |
| 1 | 2 | 3 | 2078.92 | 1905.30 | 41.09 | 35.96 | 142.35 | 172.47 | .8 | .1 |
| 2 | 3 | 2 | 2069.60 | 1843.50 | 31.59 | 26.39 | 113.9 | 123.78 | .5 | .7 |

The average daily gain in group 1 (Table 12) over a 14-day breeding period was 22.35 g, which was higher than the result obtained for same age chicks from group 2.

Over the 28-day period, the average daily gain in group 1 was 3.64% higher than in group 2.

As estimated for the 35-day breeding period, the average daily gain in group 1 was 3.28% higher than in group 2. The average daily gain in 37-day-old broilers from group 1 was increased by 1.95% as compared to same age chicks from group 2.

The average daily gain in 37-day-old cockerels from group 1 was almost identical to that value in group 2, this parameter of same age pullets from group 1 increased as compared to female chicks from group 2.

TABLE 5
Average daily gain in broiler chickens (g)

| | Age, days | | | | | | | |
|---|---|---|---|---|---|---|---|---|
| | | | 35 | | | 37 | | |
| | | | Aggre- | Fe- | | Aggre- | Fe- | |
| Group | 14 | 28 | gate | male | Male | gate | male | Male |
| 1 | 22.35 | 43.57 | 50.95 | 50.34 | 52.12 | 51.81 | 50.21 | 54.90 |
| 2 | 22.10 | 42.04 | 49.33 | 47.68 | 52.13 | 50.82 | 48.55 | 54.66 |

TABLE 6
Survival rate in Cobb 500 broiler chickens, %

Age, days

| Group | 7 | 14 | 21 | 28 | 35 | 37 |
|---|---|---|---|---|---|---|
| 1 | 100 | 100 | 100 | 100 | 100 | 100 |
| 2 | 100 | 100 | 100 | 100 | 100 | 100 |

The food conversion estimate (feed intake per 1 kg gain) (Table 7) in broilers over a 35-day period was 4.88% lower in group 1 as compared to group 2.

TABLE 7
Food conversion measurements (per 1 kg weight
gain) in broilers over 35-day period, kg.

Group

| Age, days | 1 | 2 |
|---|---|---|
| 35 | 1.56 | 1.64 |
| 37 | 1.64 | 1.70 |

The food intake per 1 kg of weight gain (food conversion) in group 1 as estimated for 37 days was 1.64 kg. This value was 3.53% lower in group 1 than in group 2.

The obtained results of the study stock were used to calculate the European broiler Index for Cobb 500 cross at 35 and 37 days of life, the figures given in Table 8.

TABLE 8
European broiler index in Cobb 500 broiler chickens

Age, days

| Group | 35 | 37 |
|---|---|---|
| 1 | 335.34 | 323.80 |
| 2 | 309.02 | 306.43 |

As shown in Table 15, the broiler index of group 1 was higher by 26.32 and 17.37 than that in the control group, as estimated at 35 and 37 days respectively.

There were no pathological findings in the examined parameters of complete blood count and biochemical analysis of the blood samples from either of the broiler groups at 37 days of life, which highlights high safety of the claimed compound for promoting weight gain in mammals and birds, comprising an activated-potentiated form of an antibody to human interferon gamma and an activated-potentiated form of an antibody to the insulin receptor β-subunit.

### Example 3

The effect of the claimed compound drug for promoting body weight gain in mammals and birds, enhancing the effectiveness of immunization, and preventing and/or treating infectious diseases, in the form of aqueous solution containing an activated-potentiated form of antigen-purified ultra-low dose antibody to human interferon gamma (prepared by extreme dilution of the primary matrix solution (concentration of 2.5 mg/ml) by a factor of 100¹², 100³⁰, 100⁵⁰), which is equivalent to a mixture of centesimal homeopathic C12, C30 and C50 dilutions (anti-IRβ Ab), on livestock performance was evaluated in floor-housed (males and females together) Cobb 500 broiler chickens in the settings of full-scale breeding facility Novorossiysk Poultry Farm.

The testing was performed in 2 facility units: the first batch of day-old broiler chicks (n=25720) was housed in Unit 1 and received the compound drug prepared from a powder comprising affinity-purified antibody to interferon gamma in a RA (release-active) form** (RA anti-IFNγ) and affinity-purified antibody to a C-terminal fragment of the insulin receptor β-subunit* in a RA form (RA anti-IRβ).

* applied onto lactose monohydrate as a mixture of 3 active aqueous-alcoholic dilutions of the substance—diluted by a factor of 100¹², 100³⁰, 100²⁰⁰, respectively;
** applied onto lactose monohydrate as a mixture of 3 active aqueous-alcoholic dilutions of the substance—diluted by a factor of 100¹², 100³⁰, 100⁵⁰, respectively;

The other batch of day-old broilers (n=25900) was housed for fattening in Unit 2 and given Placebo (lactose monohydrate). The study treatments prepared as water solutions were administered to the chickens beginning from day-old. To prepare the sample solution, the required amount of powder (g) was diluted in an appropriate volume of water (I) as shown in Table 9. The solution was repeatedly shaken to ensure homogenous dissolution; after which it was fed to the water supply system so that the chicks could receive it with drinking water. The birds were to receive the prepared solution with drinking water through a medicator for at least 3 hours irrespective of broilers' age. During the rest of the time the broilers were given clean tap water. On days of scheduled vaccination or treatment administration, drinking water mixed with the study preparations was given to chickens separately—in the afternoon; on any other days the study treatments were administered in the morning. Before being transported for slaughter or marketing, the chickens in the unit of study treatment received the preparation through day 42, and day 45—in the placebo unit.

TABLE 9

| Unit dose weight | Diluent volume | Number of |
|---|---|---|
| (powder), g | (water), L | chicks housed |
| 50 | 1 | up to 14 999 |
| 100 | 2 | between 15 000 and 24 999 |
| 150 | 3 | between 25 000 and 34 999 |

Boiler vaccination was performed using the in-house schedule:

infectious bronchitis vaccination (in the hatchery at 1 and 6 days of life) by coarse aerosol spraying (dry live Nobilis IB Ma5 vaccine, Intervet International By, Netherlands) and via drinking water (dry live IB H-120 vaccine, Pokrosysky Biologics Plant, JSC);

infectious bursal disease (IBD) vaccine (dry live; Pokrosysky Biologics Plant, JSC)—at 8 and 17 days, via drinking water;

Newcastle disease vaccine (dry live NB LaSota vaccine; Pokrosysky Biologics Plant, JSC)—at 13 days of life, via drinking water.

During the testing period, the following parameters were recorded:

1. Body weights on days 1, 7, 14, 21, 28, 35 and 42 of life for 100 chicks from each unit. The broilers were selected randomly from the reared flock and weighed individually on an electronic balance;
2. Average daily weight gain of the broilers over a 42-day breeding period (aggregate female and male data), based on weight measurements for 100 randomly selected broilers from each unit;
3. Survival rate (%) of the broiler flock—by recording the daily number of deaths and specifying causality;
4. Food consumption in each unit was estimated for the whole broiler stock and measured every 2-3 days when receiving truck deliveries of combined feed. The food intake (kg) (food conversion) per 1 kg of weight gain was calculated as: Food intake (kg)/Body weight gain (kg)
5. Following slaughter, 6 broilers from each unit (3 males and 3 females) were autopsied, and the following parameters were assessed: gross appearance of the internal organs, half-eviscerated carcass weight, eviscerated carcass weight, organ weight and condition (liver, heart, gizzard stomach, lungs and kidney).

The statistical analysis of study results was based on the following assumptions:

1. the power of statistical tests, P=(1−β), was defined as 80% (probability of correct rejection of the null hypothesis was 0.8)
2. the acceptable probability of type 1 error (a) was not greater than 5% (probability of incorrectly accepted alternative hypothesis was less than 0.05);
3. the analysis relied on two-tailed tests due to the lack of post-hoc data to demonstrate differences in the efficacy of two treatments under comparison.

For characterization of the broiler samples, the studied variables were presented as the mean (M) and standard deviation (SD).

To verify differences between the groups, the following series of tests were utilized:

1. T-test in two variants depending on the homogeneity of variances checked using the Fisher test to compare the groups in case of normal data distribution;
2. Repeated Measures ANOVA or GLMM (Generalized Linear Mixed Models) test to implement comparison taking into account changes in the variables over time;
3. Mann-Whitney test in case of skewed distribution;
Chi-square test, the Fisher exact test (if one of the observed frequencies was lower than 5), or the Cochran-Mantel-Haenszel test for frequency analysis.

Body weight data of 100 randomly selected broilers are given in Table 2. The body weights of broilers in the unit of study treatment at 42 days of monitoring were significantly increased as compared to same age broilers in the unit of placebo. The difference in this parameter between the units was 100.9 g, i.e. 5.7%.

TABLE 10
Broiler body weights on days 7, 14, 21, 28, 35 and 42 of the breeding period.

| Unit | Day 7 | Day 14 | Day 21 | Day 28 | Day 35 | Day 42 |
|---|---|---|---|---|---|---|
| Treatment | 143.8 ± 17.2 | 410.3 ± 59.2 | 678.6 ± 160.5 | 1286.6 ± 160.7 | 1611.8 ± 222.5 | 1862.1 ± 203.6* |
| Placebo | 130.4 ± 15.5 | 325.4 ± 65.8 | 653.3 ± 139.0 | 1236.5 ± 159.1 | 1614.3 ± 180.3 | 1761.2 ± 203.7 |

Note:
the results of two-factor analysis of variance of broiler body weight data: ‘Unit’ factor (2-level) and ‘Day’ factor (6-level).
1. Analysis of variance yielded a significant difference in the Unit factor F(1/1188) = 27.5, p < 0.0001;
2. Analysis of variance yielded a significant difference in the Day factor F(5/1188) = 4194, p < 0.0001;
3. Analysis of variance yielded a significant Unit*Day interaction F(5/1188) = 3.7 p = 0.0023
4. Post-Hoc analysis of the Unit*Day interaction (indicates difference between Units on a particular day) with the use of the Scheffe test only demonstrates a significant difference between the study treatment and placebo on day 42 (p = 0.0206).

Average daily gain values over the whole testing period calculated as: weight gain over the breeding period/feed days (where feed days are the total number of days in the breeding period when all reared broiler chickens are available, with account of losses: deaths, broilers slaughtered or delivered for marketing, etc) were comparable between the units of study treatment and placebo—41.58 and 41.64, respectively.

Furthermore, the test compound demonstrated prophylactic efficacy against chicken bacterial infection, which, as a result, made it possible to considerably increase the livestock survival rate. There was an increase in broiler mortality recorded during the monitoring period. The highest mortality was observed in the second half of the breeding programme; i.e., the average daily number of deaths from day 33 through day 40 was 122 in the unit of study treatment, whereas the average daily loss in the unit of placebo was 136.6 broilers over the period from day 26 through 42. Besides, there was an increment in mortality recorded in the unit of placebo from day 10 through 12, with the average daily loss of 140 broilers.

General bacteriological examination detected Proteus mirabilis and Escherichia coli in broilers from the unit of study treatment. In the examination of pathological changes in broilers from the placebo unit, Salmonella pullorum, Escherichia coli and Proteus mirabilis were detected by general analysis. Differentiated examination in placebo-treated chickens indicated the presence of the following bacteria in examined organs: Streptococcus avium and Escherichia coli (intestines), Escherichia coli (brain), Salmonella pullorum (long bones), Proteus mirabilis (heart, spleen and liver). Appropriate therapeutic activities were undertaken in both units to combat infections. High mortality in the placebo group required administering an antibiotic drug (Inflox) at a dosage of 1 ml/l for 7 days; in the group of study treatment, inflox was given at 1 ml/l (preventive regimen) and discontinued after 3 days due to the positive effect of the test compound.

In the unit of study treatment, the mortality figure was 2068 out of 25720 broilers placed for fattening, whereas 3764 out of 25900 broilers were lost in the unit of placebo. The mortality and survival rates were finally estimated to be 8.04% and 91.96% in the unit of study treatment, respectively, and 14.54% and 85.46%, respectively, in the placebo unit. Thus, the survival of broilers in the unit of study treatment was 6.5% higher than that in the unit of placebo.

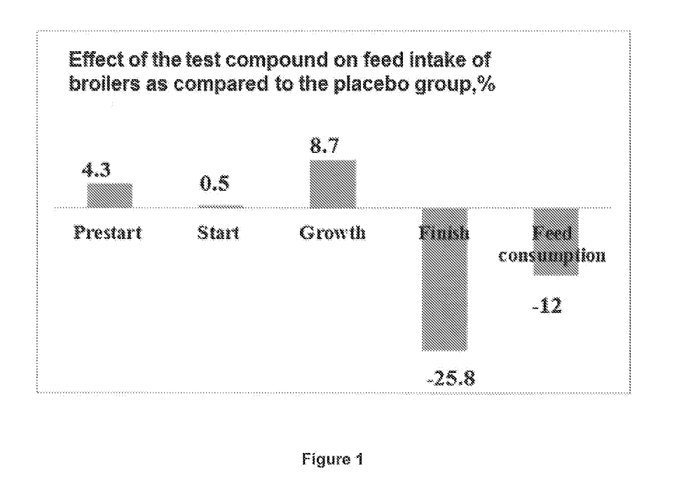

FIG. 1 displays feed intake values in the breeding units. Notably, the rates of consumption of different feed types were different in the units. The total feed consumption in the unit of study treatment was 12830 kg lower, i.e. 12%, as compared to the placebo unit.

The feed conversion estimate (intake per 1 unit weight gain) was 2.2 in the unit of study treatment and 2.43 in the unit of placebo, which is indicative of higher feed efficiency in the first unit.

The data of post-mortem examination of 6 broilers from each group (3 females and 3 males) did not reveal any significant differences in assessed parameters in comparison between the breeding units. The internal organs were normal and had no pathological changes. However, the eviscerated carcass yield in the unit of study treatment (from males and females) was 0.7% and 0.9% higher, respectively, than in the unit of placebo, which appears to be an important production efficiency indicator.

### Example 4

The effect of the claimed compound drug formulated as aqueous solution containing affinity-purified antibody to human interferon gamma (anti-IFNγ Ab) in a RA form—a mixture of 3 active aqueous dilutions of the substance diluted by a factor of 100¹², 100³⁰, 100⁵⁰, respectively, and affinity-purified antibody to a C-terminal fragment of the insulin receptor β-subunit (anti-IRβ Ab) in a release-active form—a mixture of 3 active water dilutions of the substance diluted by a factor of 100¹², 100³⁰, 100²⁰⁰, respectively, on broiler performance was evaluated at the facility of VNITIP under the Russian Agricultural Academy (town of Sergiev Posad). The production testing was performed on Cobb 500 boilers reared in R-15 battery brooders (35 co-housed male and female chickens, from 1 to 37 days of age, in each cage).

The testing was conducted in accordance with the Guidelines for Broiler Meat Production (Sergiev Posad, 2008) and Guidance on Research and Production Studies of Poultry Feeding Practices (Sergiev Posad, 2013). The assessed parameters in the group of study treatment (test compound) were compared with those in a placebo group receiving distilled water. Data on study design used for the production testing is given in Table 11.

TABLE 11

| | Number | | Daily | Route of | |
|---|---|---|---|---|---|
| | of | | dosage, | adminis- | |
| Group | chicks | Treatment | ml/chick | tration | Regimen |
| 1 | 140 | Distilled | 20 | with | twice daily |
| | | water | | drinking | (10 ml/ |
| | | | | water | chick × 2), |
| | | | | | from day 7 |
| | | | | | through day 37 |
| | | | | | of age |
| | | | | | (30 days) |
| 2 | 140 | Compound | 20 | with | twice daily |
| | | | | drinking | (10 ml/ |
| | | | | water | chick × 2), |
| | | | | | from day 7 |
| | | | | | through day 37 |
| | | | | | of age |
| | | | | | (30 days) |

Vaccination of broiler chickens was performed using the following schedule:
at hatchery (day-old chicks)—against Newcastle disease virus, infectious bronchitis (aerosol exposure), and Marek's disease (neurolymphomatosis) (intramuscularly);
at 13 days of age—against infectious bursal disease (IBD) (via drinking water);
at 19 days of age—repeated vaccine against Newcastle disease virus and infectious bronchitis (aerosol exposure);

For the vaccination procedure, the following vaccines were used: dry live vaccine against avian Newcastle disease virus (ND), strain LaSota, produced by VNIVIP of the Russian Academy of Agricultural Sciences; dry live vaccine against avian infectious bronchitis virus (IB) prepared from H-120 strain, supplied by Cronvet, Ltd.; dry live ‘Avivak-IBD’ vaccine against infectious bursal disease virus (IBD), strains Winterfield 2512 or BK, supplied by Avivak, JSC.

The following test parameters were examined:

1. Body weights of all birds under study as examined on days 1, 7 and 37 of life;

2. Average daily gain in broilers over a 37-day breeding period (both in aggregate and for separate sexes);

3. Survival rate (%) in the broiler flock—by recording the daily number of deaths and specifying causality;

4. Daily food consumption in the groups and feed conversion (feed intake per 1 kg weight gain) as calculated over a 37-day breeding period.

$$Food conversion = \frac{Feed consumed ( kg )}{Weight gain ( kg )}$$

5. Based on the growth performance figures, European broiler index (EBI) was calculated for each group.

$$EBI = \frac{Live weight ( kg ) \times Livability ( \% )}{Fattening period ( days ) \times Feed conversion ( kg \text{/} kg )} \times 100 \%$$

6. Following slaughter, meat yield data were collected at the end of the breeding period as well as carcass yield estimates by quality categories in accordance with government industry standard GOST R 52702-2006.

7. The slaughtered broilers were exposed to postmortem examination, where the following parameters were analyzed (6 broilers from each group: 3 males and 3 females)
gross examination data;
half-eviscerated weight;
eviscerated weight;
organ weight and condition (liver, heart, gizzard stomach, lungs, and kidney).

The statistical analysis of study results was based on the following assumptions:
the power of statistical tests, P=(1−β), was defined as 80% (probability of correct rejection of the null hypothesis was 0.8);
the acceptable probability of type 1 error (α) was not greater than 5%
(probability of incorrectly accepted alternative hypothesis was less than 0.05);
the analysis relied on two-tailed tests due to the lack of post-hoc data to demonstrate differences in the efficacy of two treatments under comparison.

For characterization of the broiler samples, the following variables were calculated:
the mean (M);
the standard deviation (SD).
To verify differences between the groups, the following series of tests were utilized:
Repeated Measures ANOVA or GLMM (Generalized Linear Mixed Models) test to implement comparison taking into account changes in the variables over time;
T-test in two variants depending on the homogeneity of variances checked using the Fisher test to compare the groups in case of normal data distribution;
Mann-Whitney test in case of skewed distribution;
Normality verification was performed with the use of the Kolmogorov-Smirnov-Lilliefors test.
Chi-square test, the Fisher exact test (if one of the observed frequencies was lower than 5), or the Cochran-Mantel-Haenszel test for frequency analysis.
The productivity results obtained in the study groups of Cobb 500 broilers are presented in Table 12.

TABLE 12
Body weights of broiler chickens from day 1 to day 37, g

| | Days: | | | | |
|---|---|---|---|---|---|
| | | | Δ day 7 | | Δ day 37 |
| | | | compared to | | compared to |
| | 1 | 7 | baseline | 37 | baseline |
| Control | 51.1 ± 2.5 | 127.8 ± 8.7 | 76.79 | 1880.1 ± 138.7 | 1829.07 |
| | n = 140 | n = 140 | | n = 138 | |
| Test | 50.7 ± 2.4 | 128.8 ± 11.1 | 78.04 | 1914.3 ± 155.1 | 1863.52 |
| compound | n = 140 | n = 140 | | n = 140 | |
| Δ between | 0.31 | −0.94 | −1.26 | −34.14 | −34.46 |
| groups | | | | | |

The productivity analysis of broiler chickens using the two-factor analysis of variance (ANOVA; Group factor (2-level) and Day factor (3-level) yielded:

1. a significant difference in Group factor: F(1/832)=3.86; p=0.0498;
2. a significant difference in Day factor: F(2/832)=42076.4; p<0.0001;
3. a significant difference in Treatment-Visit interaction—F(2/832)=3.68; p=0.0256.

Moreover, post-hoc analysis using the Bonferroni test did not reveal significant differences between the groups of day-old (p=1.0) and 7-day-old chickens (p=1.0), while the broiler body weight data in the group of study treatment as collected at 37 days of the breeding period and evaluated using the Bonferroni adjustment exceeded significantly the corresponding values in the control group (p=0.0127), with the difference between the groups of 34.14 g.

The average weight gain per day (calculated as difference in mean body weights of 37-day-old and day-old chickens divided by the number of feeding days (37 days)) was increased in the group of test compound as compared to that in the control group. The average daily gain of controls was 49.41 g, and this value in the group of test compound was 50.37 g.

Notably, birds in the group of test compound demonstrated a high survival rate: 100% of broilers survived to the end of the study, while the loss rate in the control group was 1.4%, i.e. the percentage of broilers surviving to the end of the study was 98.6%.

Although the food conversion was comparable in both groups (i.e., 1.5 kg feed per 1 kg gain), the European broiler index in the group of test compound was 343.7, and 336.2 in the control group. Thus, the difference between the groups was 7.5 (i.e., 2.2%).

Data for the whole tested broiler stock were used to assess the effects of the study treatments on meat yield and carcass quality by categories. The results are given in Table 13.

The total carcass weights in the control group and group of test compound were 186.28 kg and 194.81 kg, respectively; mean weights per carcass in these groups were 1349 g and 1392 g, respectively. Thus, the carcass weight in the group of test compound was increased by 3.2% as compared to that in the control group.

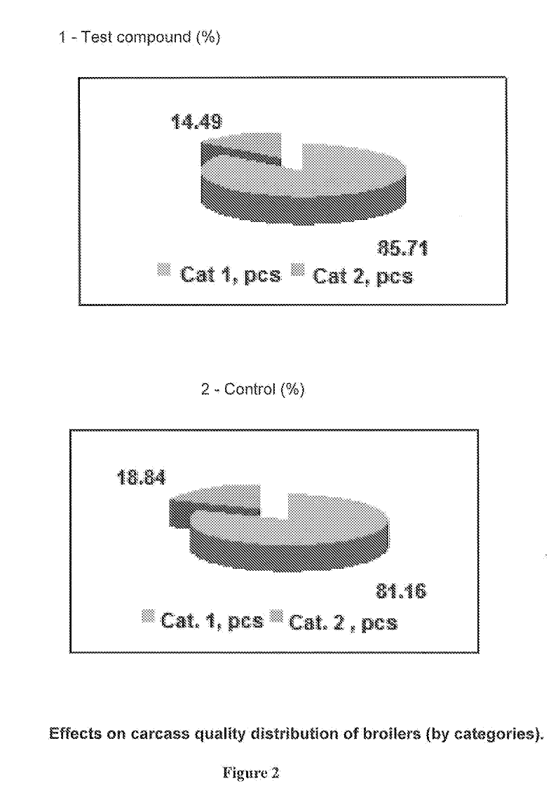

The total meat yield in the broiler group treated with test compound was 4.6% higher than in the control group. Quality grading of broiler carcasses showed that the number of Category 1 carcasses in the group of test compound was 9% greater as compared to controls (FIG. 2).

TABLE 13
Meat yield and quality categories of broilers.

| Parameter | Control | Compound |
|---|---|---|
| Number of carcasses | 138 | 140 |
| Including Category 1 carcasses, n | 112 | 120 |
| Including Category 2 carcasses, n | 26 | 20 |
| Yield Category 1, kg | 156.32 | 170.52 |
| Yield Category 2, kg | 29.96 | 24.29 |
| Total carcass weight, kg | 186.28 | 194.81 |

The analysis of data obtained from postmortem examination of 6 broilers from each group (3 males and 3 females) showed that the internal organs were normal, without pathological changes. Half-eviscerated carcass yield did not differ significantly in the two groups; however, an increasing trend in half-eviscerated weight data was observed in the group of test compound as compared to the control group (p=0.052). Eviscerated yield is of more importance as broiler meat is marketed nowadays as eviscerated carcasses and carcass parts, as defined by GOST R 527-2006. The eviscerated meat yield was significantly increased in the compound-treated group as compared to controls—by 3.6%. The number of eviscerated carcasses yielded by the group of test compound as related to the broiler body weight in the same group was 74.2%, which was 1.3% higher than in the control group, where this value was 72.9%.

### Example 5

The effect of the claimed compound drug formulated as aqueous solution containing affinity-purified antibody to human interferon gamma (anti-IFNγ Ab) in a RA form—a mixture of 3 active water dilutions of the substance diluted by a factor of 100¹², 100³⁰, 100⁵⁰, respectively), and affinity-purified antibody to a C-terminal fragment of the insulin receptor β-subunit (anti-IRβ Ab) in a release-active form—a mixture of 3 active water dilutions of the substance, diluted by a factor of 100¹², 100³⁰, 100²⁰⁰, respectively) on the immune status of broiler chickens and effectiveness of immunization was evaluated at the facility of VNITIP under the Russian Agricultural Academy (town of Sergiev Posad). The production testing was performed on Cobb 500 boilers reared in R-15 battery brooders (35 co-housed male and female chickens, from 1 to 37 days of age, in each cage).

The rearing of broilers complied with the Guidelines for Broiler Meat Production (Sergiev Posad, 2008) and Guidance on Research and Production Studies of Poultry Feeding Practices (Sergiev Posad, 2013). The assessed parameters in the group of study treatment (test compound) were compared with those in a placebo group receiving distilled water. Data on study design used for the production testing is given in Table 14.

TABLE 14
Production testing particulars

| | Number | | Daily | Route of | |
|---|---|---|---|---|---|
| | of | | dosage, | adminis- | |
| Group | chicks | Treatment | ml/chick | tration | Regimen |
| 1 | 140 | Distilled | 20 | with | twice daily |
| | | water | | drinking | (10 ml/ |
| | | | | water | chick × 2), |
| | | | | | from day 7 |
| | | | | | through day 37 |
| | | | | | of age |
| | | | | | (30 days) |
| 2 | 140 | Compound | 20 | with | twice daily |
| | | | | drinking | (10 ml/ |
| | | | | water | chick × 2), |
| | | | | | from day 7 |
| | | | | | through day 37 |
| | | | | | of age |
| | | | | | (30 days) |

Vaccination of broiler chickens was performed using the following schedule:
at hatchery (day-old chicks)—against Newcastle disease virus, infectious bronchitis (aerosol exposure), and Marek's disease (neurolymphomatosis) (intramuscularly);
at 13 days of age—against infectious bursal disease (IBD) (via drinking water);
at 19 days of age—repeated vaccine against Newcastle disease virus and infectious bronchitis (aerosol exposure);

For the vaccination procedure, the following vaccines were used: dry live vaccine against avian Newcastle disease virus (ND), strain LaSota, produced by VNIVIP of the Russian Academy of Agricultural Sciences; live dry vaccine against avian infectious bronchitis virus (IB) prepared from H-120 strain, supplied by Cronvet, Ltd.; dry live ‘Avivak-IBD’ vaccine against infectious bursal disease virus (IBD), strains Winterfield 2512 or BK, supplied by Avivak, JSC.

Serological testing was performed at slaughter in 37-day-old broilers. The blood samples were derived from 30 chickens in each group under comparison and delivered for further analysis to the Russian National Centre for Standardization and Testing of Veterinary Drugs and Feeds (VGNKI) (Moscow, Russia).

The samples were tested for antibodies to avian infectious bronchitis virus (IBV)—using ELISA assay, antibodies to Newcastle disease virus—using a hemagglutination inhibition test, and antibodies to Gumboro disease virus (infectious bursal disease, IBD)—by ELISA assay.

The Mann-Whitney test was used for statistical analysis of test results.
The results of serological testing are presented in Table 15.

TABLE 15
Effects of study treatments on antibody titer

| | Mean antibody titer/% positive probes | | |
|---|---|---|---|
| Group | NDV (HI test) | IBV (ELISA) | IBD (ELISA) |
| Control | 5.6 ± 1.9 log2/100 | 1:1400/77 | 1:140/3 |
| Compound | 6.4 ± 1.8 log2/100 | 1:2331/73 | 1:234/10* |

Note:
*p < 0.05 as evaluated by the Mann-Whitney U-test

The analysis of quantitative data regarding virus-specific antibodies in broiler blood serum showed that the effectiveness of immunization was increased as a result of test compound administration. Thus, the specific antibody titer against NDV and IBD in the compound-treated group was higher than in the control group, with significant differences between the groups in the case of mean anti-IBD titer. The test compound demonstrated a somewhat lower protective efficacy against infectious bronchitis virus.

### Example 6

The study investigated the effect of the claimed compound drug formulated as aqueous solution containing release-active affinity-purified antibody to human interferon gamma (anti-IFNγ Ab)—a mixture of 3 active aqeous dilutions of the substance diluted by a factor of 100¹², 100³⁰, 100⁵⁰, respectively; release-active affinity-purified antibody to a C-terminal fragment of the insulin receptor β-subunit (anti-IRβ Ab)—a mixture of 3 active aqueous dilutions of the substance diluted by a factor of 100¹², 100³⁰, 100²⁰⁰, respectively; and release-active affinity-purified antibody to CD4 receptor (anti-CD4 Ab)—a mixture of 3 active aqueous dilutions of the substance diluted by a factor of 100¹², 100³⁰, 100⁵⁰, respectively, on broiler performance.

The test compound was administered with drinking water over the following periods: from day 1 to 5, from day 17 to 21, from day 27 to 31 (Group 1)—or daily from day 1 to 37 (Group 2).

The control birds (Group 3) received clean drinking water.
Each study group comprised a total of 452 000 broiler chickens.

The following parameters were examined within the study:

1. Broilers' body weights on days 7, 14, 21, 28, 35 and 37 of life;
2. Average weight gain of the broilers per day;
3. Survival rate (%) of the broiler flock;
4. Food consumption per week;
5. Feed conversion over the whole study period;
6. European Production Efficiency Factor calculated for broiler groups
7. Peripheral blood variables on days 5, 11, 22 and 35.

According to the study data, both periodic (short-term) and continuous administration of the compound drug with drinking water resulted in a sustainable increase in broiler performance (the obtained results are given in Table 16). The average weight gain per day was 59.01 g in Group 1 and 59.37 g in Group 2 (this value in the control group was 57.7 g). Moreover, the body weights of all drug-treated birds were averagely 80-90 g higher that the corresponding control values. The lowest feed conversion results (feed intake per 1 kg weight gain) were obtained in the groups of drug treatment. The flock livability with both short-term and continuous drug administration was increased by 5.07% and 3.32%, respectively. The production efficiency factor was substantially improved as well.

TABLE 16
Broiler performance results in different study groups.

| | | | | | |
|---|---|---|---|---|---|
| | | Average | | | |
| Broiler | Average | live | | | |
| production | daily | weight, | Livability | | Efficiency |
| unit ID | gain, g | kg | % | Conversion | factor |

Group 1: test compound administered with drinking water over
separate time periods

| | | | | | | |
|---|---|---|---|---|---|---|
| 60 | PV_15_{—} | 60.74 | 2.31 | 90.22 | 1.77 | 315 |
| 60 | PV_16_{—} | 60.18 | 2.26 | 91.08 | 1.75 | 318 |
| 60 | PV_17_{—} | 58.09 | 2.18 | 91.34 | 1.86 | 290 |
| 60 | PV_18_{—} | 60.34 | 2.21 | 96.74 | 1.73 | 343 |
| 60 | PV_19_{—} | 63.04 | 2.3 | 96.98 | 1.67 | 372 |
| 60 | PV_20_{—} | 60.15 | 2.2 | 91.34 | 1.85 | 301 |
| 60 | PV_21_{—} | 60.1 | 2.2 | 91.35 | 1.78 | 314 |
| 60 | PV_22_{—} | 57.45 | 2.1 | 91.3 | 1.86 | 286 |
| 60 | PV_23_{—} | 56.01 | 2.11 | 94.16 | 2.08 | 258 |
| 60 | PV_24_{—} | 58.96 | 2.2 | 97.46 | 1.92 | 304 |
| 60 | PV_25_{—} | 58.71 | 2.15 | 95.89 | 1.81 | 316 |
| 60 | PV_26_{—} | 58.61 | 2.15 | 97.57 | 1.78 | 326 |
| 60 | PV_27_{—} | 59.1 | 2.22 | 93.39 | 1.87 | 296 |
| 60 | PV_28_{—} | 59.72 | 2.19 | 89.22 | 1.92 | 277 |

| | | | | | |
|---|---|---|---|---|---|
| Mean | 59.37 | 2.199 | 93.43 | 1.83 | 308 |

Group 2: test compound administered continuously (with drinking
water) throughout the breeding period

| | | | | | | |
|---|---|---|---|---|---|---|
| 60 | PV_1_{—} | 59.15 | 2.16 | 93.49 | 1.92 | 292 |
| 60 | PV_2_{—} | 59.79 | 2.19 | 90.15 | 1.97 | 279 |
| 60 | PV_3_{—} | 60.26 | 2.21 | 95.47 | 1.85 | 317 |
| 60 | PV_4_{—} | 58.98 | 2.22 | 90.65 | 1.92 | 283 |
| 60 | PV_5_{—} | 59 | 2.22 | 95.67 | 1.88 | 305 |
| 60 | PV_6_{—} | 57.41 | 2.16 | 89.16 | 2.09 | 249 |
| 60 | PV_7_{—} | 61.3 | 2.24 | 93.88 | 1.8 | 324 |
| 60 | PV_8_{—} | 58.98 | 2.2 | 89 | 1.94 | 274 |
| 60 | PV_9_{—} | 59.35 | 2.17 | 91.58 | 1.88 | 294 |
| 60 | PV_10_{—} | 59.55 | 2.18 | 92.77 | 1.85 | 303 |
| 60 | PV_11_{—} | 59.15 | 2.16 | 96.33 | 1.72 | 336 |
| 60 | PV_12_{—} | 57.54 | 2.16 | 89.85 | 1.85 | 285 |
| 60 | PV_13_{—} | 58.48 | 2.2 | 87.29 | 1.86 | 278 |
| 60 | PV_14_{—} | 57.23 | 2.15 | 88.19 | 1.88 | 273 |

| | | | | | |
|---|---|---|---|---|---|
| Mean | 59.37 | 2.187 | 91.68 | 1.89 | 292 |

Group 3: control

| | | | | | | |
|---|---|---|---|---|---|---|
| 60 | PV_43_{—} | 56.98 | 2.09 | 95.75 | 1.88 | 295 |
| 60 | PV_44_{—} | 56.68 | 2.08 | 91.35 | 1.87 | 282 |
| 60 | PV_45_{—} | 57.68 | 2.17 | 89.99 | 1.88 | 281 |
| 60 | PV_46_{—} | 57.31 | 2.04 | 90.78 | 1.93 | 275 |
| 60 | PV_47_{—} | 58.36 | 2.08 | 93.75 | 1.78 | 313 |
| 60 | PV_48_{—} | 56.52 | 2.01 | 93.68 | 1.84 | 292 |
| 60 | PV_49_{—} | 59.38 | 2.17 | 81.81 | 2.05 | 241 |
| 60 | PV_50_{—} | 58.24 | 2.13 | 81.81 | 2.09 | 232 |
| 60 | PV_51_{—} | 58.18 | 2.19 | 83.4 | 2.01 | 245 |
| 60 | PV_52_{—} | 57.42 | 2.1 | 85.61 | 2.02 | 247 |
| 60 | PV_53_{—} | 59.56 | 2.18 | 89.43 | 1.85 | 292 |
| 60 | PV_54_{—} | 58.55 | 2.14 | 88.54 | 1.91 | 276 |
| 60 | PV_55_{—} | 57.57 | 2.06 | 87.8 | 1.98 | 260 |
| 60 | PV_56_{—} | 55.56 | 2.04 | 83.29 | 2.05 | 230 |

| | | | | | |
|---|---|---|---|---|---|
| Mean | 57.71 | 2.106 | 88.36 | 1.94 | 269 |

The study demonstrated a beneficial effect of the claimed compound drug with both short-term and continuous administration, which was seen to result in higher body weight gain of the chickens, better flock livability, and lower feed outputs required.

What is claimed is:

1. A pharmaceutical composition for use in humans, non-human animals or birds comprising a) an activated-potentiated form of an antibody to human insulin receptor and b) an activated-potentiated form of an antibody to human interferon gamma.

2. The pharmaceutical composition of claim 1, wherein said activated-potentiated form of an antibody to human insulin receptor is an activated-potentiated form of an antibody to a C-terminal fragment of the insulin receptor β-subunit.

3. The pharmaceutical composition of claim 2, wherein said activated-potentiated form of an antibody to a C-terminal fragment of the insulin receptor β-subunit and said activated-potentiated form of an antibody to human interferon gamma are in the form of an aqueous or aqueous-alcoholic solutions with the activity achieved through repeated sequential dilution of the primary matrix antibody solution in a water or alcohol-water solvent, coupled with external mechanical treatment of each dilution.

4. The pharmaceutical composition of claim 2, formulated as a solid unit dosage form and comprising a technologically required amount of a neutral carrier saturated with said combined activated-potentiated forms of an antibody to a C-terminal fragment of the insulin receptor β-subunit and an antibody to human interferon gamma along with pharmaceutically acceptable excipients.

5. The pharmaceutical composition of claim 4, wherein said aqueous or aqueous-alcoholic solutions of the activated-potentiated forms of antibodies to a C-terminal fragment of the insulin receptor β-subunit and to human interferon gamma are obtained via repeated sequential dilution of the primary matrix solutions of antibodies to a C-terminal fragment of the insulin receptor β-subunit and to human interferon gamma, coupled with vertical shaking of each dilution, said primary matrix solutions having a concentration of 0.5÷5.0 mg/ml.

6. The pharmaceutical composition of claim 1, wherein each composition component is used in the form of a mixture of centesimal dilutions obtained according to a homeopathic manufacturing methodology.

7. The pharmaceutical composition of claim 4, wherein the pharmaceutically acceptable excipients include lactose, microcrystalline cellulose and magnesium stearate.

8. The pharmaceutical composition of claim 4, wherein the pharmaceutically acceptable excipients include isomalt, sodium cyclamate, sodium saccharine, anhydrous citric acid and magnesium stearate.

9. A method of improving livability of food-producing animals, non-human mammals or birds, said method comprising administering to said animal, non-human mammal or bird an activated-potentiated form of an antibody to human insulin receptor and an activated-potentiated form of an antibody to human interferon gamma.

10. The method of claim 9 comprising administering to the animal an activated-potentiated form of an antibody to a C-terminal fragment of the insulin receptor β-subunit and an activated-potentiated form of an antibody to human interferon gamma.

11. The method of claim 10, wherein said activated-potentiated forms of antibodies to a C-terminal fragment of the insulin receptor β-subunit and to human interferon gamma are each used in the form of an aqueous or aqueous-alcoholic solution with the activity achieved through repeated sequential dilution of the primary matrix solutions of antibodies to a C-terminal fragment of the insulin receptor β-subunit and to human interferon gamma, respectively, in a water or alcohol-water solvent, coupled with external mechanical treatment of each dilution.

12. The method of claim 9, wherein a mixture of homeopathic dilutions of antibodies to a C-terminal fragment of the insulin receptor β-subunit and to human interferon gamma is used as unit dosage form.

13. A method of promoting body weight gain in non-human mammals or birds, said method comprising administering to said non-human mammal or bird an activated-potentiated form of an antibody to the insulin receptor and an activated-potentiated form of an antibody to human interferon gamma.

14. The method of claim 13 comprising administering an activated-potentiated form of an antibody to a C-terminal fragment of the insulin receptor β-subunit and an activated-potentiated form of an antibody to human interferon gamma.

15. The method of claim 14, wherein said activated-potentiated forms of antibodies to a C-terminal fragment of the insulin receptor β-subunit and to human interferon gamma are each used in the form of an aqueous or aqueous-alcoholic solution with the activity achieved through repeated sequential dilution of the primary matrix solutions of antibodies to a C-terminal fragment of the insulin receptor β-subunit and to human interferon gamma, respectively, in a water or alcohol-water solvent, coupled with external mechanical treatment of each dilution.

16. The method of claim 14, wherein a mixture of homeopathic dilutions of antibodies to a C-terminal fragment of the insulin receptor β-subunit and to human interferon gamma is used as a unit dosage form.

17. A method of enhancing the effectiveness of immunization in non-human mammals or birds, said method comprising administering to said non-human mammal or bird an activated-potentiated form of an antibody to the insulin receptor and an activated-potentiated form of an antibody to human interferon gamma.

18. The method of claim 17 comprising administering an activated-potentiated form of an antibody to a C-terminal fragment of the insulin receptor β-subunit and an activated-potentiated form of an antibody to human interferon gamma.

19. The method of claim 18, wherein said activated-potentiated forms of antibodies to a C-terminal fragment of the insulin receptor β-subunit and to human interferon gamma are each used in the form of an aqueous or aqueous-alcoholic solution with the activity achieved through repeated sequential dilution of the primary matrix solutions of antibodies to a C-terminal fragment of the insulin receptor β-subunit and to human interferon gamma, respectively, in a water or alcohol-water solvent, coupled with external mechanical treatment of each dilution.

20. The method of claim 18, wherein a mixture of homeopathic dilutions of antibodies to a C-terminal fragment of the insulin receptor β-subunit and to human interferon gamma is used as a unit dosage form.

21. A method of preventing and/or treating infectious diseases of non-human mammals or birds, said method comprising administering to said non-human mammal or bird an activated-potentiated form of an antibody to the insulin receptor and an activated-potentiated form of an antibody to human interferon gamma.

22. The method of claim 21 comprising administering an activated-potentiated form of an antibody to a C-terminal fragment of the insulin receptor β-subunit and an activated-potentiated form of an antibody to human interferon gamma.

23. The method of claim 22, wherein said activated-potentiated forms of antibodies to a C-terminal fragment of the insulin receptor β-subunit and to human interferon gamma are each in the form of an aqueous or aqueous-alcoholic solution with the activity achieved through repeated sequential dilution of the primary matrix solutions of antibodies to a C-terminal fragment of the insulin receptor β-subunit and to human interferon gamma, respectively, in a water or alcohol-water solvent, coupled with external mechanical treatment of each dilution.

24. The method of claim 22, wherein a mixture of various homeopathic dilutions of antibodies to a C-terminal fragment of the insulin receptor β-subunit and to human interferon gamma is used as a unit dosage form.

25. The pharmaceutical composition of claim 1, further comprising an activated-potentiated form of an antibody to CD4 receptor.

26. The pharmaceutical composition of claim 25, wherein said activated-potentiated form of an antibody to a C-terminal fragment of the insulin receptor β-subunit, said activated-potentiated form of an antibody to human interferon gamma and said activated-potentiated form of an antibody to CD4 receptor are each used in the form of an aqueous or aqueous-alcoholic solution with the activity achieved through repeated sequential dilution of the primary matrix antibody solution in a water or alcohol-water solvent, coupled with external mechanical treatment of each dilution.

27. The pharmaceutical composition of claim 25, formulated as a solid unit dosage form and comprising a technologically required amount of a neutral carrier saturated with combined activated-potentiated form of an antibody to a C-terminal fragment of the insulin receptor β-subunit, activated-potentiated form of an antibody to human interferon gamma and activated-potentiated form of an antibody to CD4 receptor, along with pharmaceutically acceptable excipients.

28. The pharmaceutical composition of claim 26, wherein the aqueous or aqueous-alcoholic solutions of the activated-potentiated forms of antibodies to a C-terminal fragment of the insulin receptor β-subunit, to human interferon gamma, and to CD4 receptor are obtained via repeated sequential dilution of the primary matrix solutions of antibodies to a C-terminal fragment of the insulin receptor β-subunit, to human interferon gamma, and to CD4 receptor, respectively, coupled with vertical shaking of each dilution, each primary matrix solution having a concentration of 0.5÷5.0 mg/ml.

29. The pharmaceutical composition of claim 25, wherein each composition component is used in the form of a mixture of centesimal, dilutions obtained according to a homeopathic manufacturing methodology.

30. The pharmaceutical composition of claim 27, wherein the pharmaceutically acceptable excipients include lactose, microcrystalline cellulose and magnesium stearate.

31. The pharmaceutical composition of claim 27, wherein the pharmaceutically acceptable excipients include isomalt, sodium cyclamate, sodium saccharine, anhydrous citric acid and magnesium stearate.

32. A method of improving livability of food-producing animals, non-human mammals or birds, said method comprising administering to said food-producing animal, non-human mammal or bird an activated-potentiated form of an antibody to the insulin receptor, an activated-potentiated form of an antibody to human interferon gamma, and an activated-potentiated form of an antibody to CD4 receptor.

33. The method of claim 32, said method comprising administering to the animal an activated-potentiated form of an antibody to a C-terminal fragment of the insulin receptor β-subunit, an activated-potentiated form of an antibody to human interferon gamma, and an activated-potentiated form of an antibody to CD4 receptor.

34. The method of claim 33, wherein said activated-potentiated forms of antibodies to a C-terminal fragment of the insulin receptor β-subunit, to human interferon gamma, and to CD4 are each used in the form of an aqueous or aqueous-alcoholic solution with the activity achieved through repeated sequential dilution of the primary matrix solutions of antibodies to a C-terminal fragment of the insulin receptor β-subunit, to human interferon gamma, and to CD4, respectively, in a water or alcohol-water solvent, coupled with external mechanical treatment of each dilution.

35. The method of claim 33, wherein a mixture of various homeopathic dilutions of antibodies to a C-terminal fragment of the insulin receptor β-subunit, to human interferon gamma, and to CD4 is used as a unit dosage form.

36. A method of promoting body weight gain in non human mammals or birds, said method comprising administering to said non-human mammal or bird an activated-potentiated form of an antibody to the insulin receptor, an activated-potentiated form of an antibody to human interferon gamma, and an activated-potentiated form of an antibody to CD4 receptor.

37. The method of claim 36 comprising administering an activated-potentiated form of an antibody to a C-terminal fragment of the insulin receptor β-subunit, an activated-potentiated form of an antibody to human interferon gamma, and an activated-potentiated form of an antibody to CD4 receptor.

38. The method of claim 37, wherein said activated-potentiated forms of antibodies to a C-terminal fragment of the insulin receptor β-subunit, to human interferon gamma, and to CD4 are each used in the form of an aqueous or aqueous-alcoholic solution with the activity achieved through repeated sequential dilution of the primary matrix solutions of antibodies to a C-terminal fragment of the insulin receptor β-subunit, to human interferon gamma, and to CD4, respectively, in a water or alcohol-water solvent, coupled with external mechanical treatment of each dilution.

39. The method of claim 37, wherein a mixture of homeopathic dilutions of antibodies to a C-terminal fragment of the insulin receptor β-subunit, to human interferon gamma, and to CD4 is used as a unit dosage form.

40. A method of enhancing the effectiveness of immunization in non-human mammals or birds, said method comprising administering to said non-human mammal or bird an activated-potentiated form of an antibody to the insulin receptor, an activated-potentiated form of an antibody to human interferon gamma, and activated-potentiated form of an antibody to CD4.

41. The method of claim 40, said method comprising administering an activated-potentiated form of an antibody to a C-terminal fragment of the insulin receptor β-subunit, an activated-potentiated form of an antibody to human interferon gamma, and an activated-potentiated form of an antibody to CD4 receptor.

42. The method of claim 41, wherein said activated-potentiated forms of antibodies to a C-terminal fragment of the insulin receptor β-subunit, to human interferon gamma, and to CD4 are each used in the form of an aqueous or aqueous-alcoholic solution with the activity achieved through repeated sequential dilution of the primary matrix solutions of antibodies to a C-terminal fragment of the insulin receptor β-subunit, to human interferon gamma, and to CD4, respectively, in a water or alcohol-water solvent, coupled with external mechanical treatment of each dilution.

43. The method of claim 41, wherein a mixture of homeopathic dilutions of antibodies to a C-terminal fragment of the insulin receptor β-subunit, to human interferon gamma, and to CD4 is used as a unit dosage form.

44. A method of preventing and/or treating infectious diseases of non-human mammals or birds, said method comprising administering to said non-human mammal or bird an activated-potentiated form of an antibody to the insulin receptor, an activated-potentiated form of an antibody to human interferon gamma, and an activated-potentiated form of an antibody to CD4.

45. The method of claim 44, comprising administering an activated-potentiated form of an antibody to a C-terminal fragment of the insulin receptor β-subunit, an activated-potentiated form of an antibody to human interferon gamma, and an activated-potentiated form of an antibody to CD4 receptor.

46. The method of claim 45, wherein said activated-potentiated forms of antibodies to a C-terminal fragment of the insulin receptor β-subunit, to human interferon gamma, and to CD4 are each used in the form of an aqueous or aqueous-alcoholic solution with the activity achieved through repeated sequential dilution of the primary matrix solutions of antibodies to a C-terminal fragment of the insulin receptor β-subunit, to human interferon gamma, and to CD4, respectively, in a water or alcohol-water solvent, coupled with external mechanical treatment of each dilution.

47. The method of claim 45, wherein a mixture of homeopathic dilutions of antibodies to a C-terminal fragment of the insulin receptor β-subunit, to human interferon gamma, and to CD4 is used as a unit dosage form.

48. The pharmaceutical composition for use in humans, non-human mammals or birds comprising an activated-potentiated form of antibody to insulin receptor and an activated-potentiated form of an antibody to CD4 receptor.

49. The pharmaceutical composition of claim 48, wherein the activated-potentiated form of an antibody to a C-terminal fragment of the insulin receptor β-subunit and said activated-potentiated form of an antibody to CD4 receptor are each used in the form of an aqueous or aqueous-alcoholic solution with the activity achieved through repeated sequential dilution of the primary matrix antibody solution in a water or alcohol-water solvent, coupled with external mechanical treatment of each dilution.

50. The pharmaceutical composition of claim 49, formulated as a solid unit dosage form and comprising a technologically required amount of a neutral carrier saturated with combined activated-potentiated form of an antibody to a C-terminal fragment of the insulin receptor β-subunit and activated-potentiated form of an antibody to CD4 receptor, along with pharmaceutically acceptable excipients.

51. The pharmaceutical composition of claim 50, wherein the aqueous or aqueous-alcoholic solutions of the activated-potentiated forms of antibodies to a C-terminal fragment of the insulin receptor β-subunit and to CD4 receptor are obtained via repeated sequential dilution of the primary matrix solutions of antibodies to a C-terminal fragment of the insulin receptor β-subunit and to CD4 receptor, respectively, coupled with vertical shaking of each dilution, each primary matrix solution having a concentration of 0.5÷5.0 mg/ml.

52. The pharmaceutical composition of claim 49, wherein each composition component is used in the form of a mixture of centesimal dilutions obtained according to a homeopathic manufacturing methodology

53. The pharmaceutical composition of claim 50, wherein the pharmaceutically acceptable excipients include lactose, microcrystalline cellulose and magnesium stearate.

54. The pharmaceutical composition of claim 50, wherein the pharmaceutically acceptable excipients include isomalt, sodium cyclamate, sodium saccharine, anhydrous citric acid and magnesium stearate.

55. A method of improving the livability of food-producing animals, non-human mammals or birds, said method comprising administering to said animal, non-human mammal or bird an activated-potentiated form of an antibody to the insulin receptor and an activated-potentiated form of an antibody to CD4 receptor.

56. The method of claim 55 comprising administering an activated-potentiated form of an antibody to a C-terminal fragment of the insulin receptor β-subunit and an activated-potentiated form of an antibody to CD4 receptor.

57. The method of claim 56, wherein said activated-potentiated forms of antibodies to a C-terminal fragment of the insulin receptor β-subunit and to CD4 are each used in the form of an aqueous or aqueous-alcoholic solution with the activity achieved through repeated sequential dilution of the primary matrix solutions of antibodies to a C-terminal fragment of the insulin receptor β-subunit and to CD4, respectively, in a water or alcohol-water solvent, coupled with external mechanical treatment of each dilution.

58. The method of claim 56, wherein a mixture of homeopathic dilutions of antibodies to a C-terminal fragment of the insulin receptor β-subunit and to CD4 is used as a unit dosage form.

59. A method of promoting body weight gain in non-human mammals or birds, said method comprising administering to said non-human mammal or bird an activated-potentiated form of an antibody to the insulin receptor and an activated-potentiated form of an antibody to CD4 receptor.

60. The method of claim 59, comprising administering an activated-potentiated form of an antibody to a C-terminal fragment of the insulin receptor β-subunit and an activated-potentiated form of an antibody to CD4 receptor.

61. The method of claim 60, wherein said activated-potentiated forms of antibodies to a C-terminal fragment of the insulin receptor β-subunit and to CD4 are each used in the form of an aqueous or aqueous-alcoholic solution with the activity achieved through repeated sequential dilution of the primary (matrix) solutions of antibodies to a C-terminal fragment of the insulin receptor β-subunit and to CD4, respectively, in a water or alcohol-water solvent, coupled with external mechanical treatment of each dilution.

62. The method of claim 60, wherein a mixture of homeopathic dilutions of antibodies to a C-terminal fragment of the insulin receptor β-subunit and to CD4 is used as a unit dosage form.

63. A method of enhancing the effectiveness of immunization in non-human mammals or birds, said method comprising administering to said non-human mammal an activated-potentiated form of an antibody to the insulin receptor and an activated-potentiated form of an antibody to CD4 receptor.

64. The method of claim 63 comprising administering to the animal an activated-potentiated form of an antibody to a C-terminal fragment of the insulin receptor β-subunit and an activated-potentiated form of an antibody to CD4 receptor.

65. The method of claim 64, wherein said activated-potentiated forms of antibodies to a C-terminal fragment of the insulin receptor β-subunit and to CD4 are each used in the form of an aqueous or aqueous-alcoholic solution with the activity achieved through repeated sequential dilution of the primary matrix solutions of antibodies to a C-terminal fragment of the insulin receptor β-subunit and to CD4, respectively, in a water or alcohol-water solvent, coupled with external mechanical treatment of each dilution.

66. The method of claim 64, wherein a mixture of homeopathic dilutions of antibodies to a C-terminal fragment of the insulin receptor β-subunit and to CD4 is used as a unit dosage form.

67. A method of preventing and/or treating infectious diseases of non-human mammals or birds, said method comprising administering to said non-human mammal or bird an activated-potentiated form of an antibody to the insulin receptor and an activated-potentiated form of an antibody to CD4.

68. The method of claim 67 comprising administering an activated-potentiated form of an antibody to a C-terminal fragment of the insulin receptor β-subunit and an activated-potentiated form of an antibody to CD4 receptor.

69. The method of claim 68, wherein said activated-potentiated forms of an antibody to a C-terminal fragment of the insulin receptor β-subunit and antibody to CD4 are each used in the form of an aqueous or aqueous-alcoholic solution with the activity achieved through repeated sequential dilution of the primary matrix solutions of antibodies to a C-terminal fragment of the insulin receptor β-subunit and to CD4, respectively, in a water or alcohol-water solvent, coupled with external mechanical treatment of each dilution.

70. The method of claim 69, wherein a mixture of homeopathic dilutions of antibodies to a C-terminal fragment of the insulin receptor β-subunit and to CD4 is used as a unit dosage form.
